(19) United States
(12) Patent Application Publication
(10) Pub. No.: US 2010/0047777 A1
(43) Pub. Date: Feb. 25, 2010

(54) METHODS FOR IDENTIFYING MUTATIONS IN CODING AND NON-CODING DNA

(75) Inventors: Aravinda Chakravarti, Lutherville, MD (US); Eileen Sproat Emison, Elkridge, MD (US); Andrew Smythe McCallion, Baltimore, MD (US); Eric Green, Bethesda, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/920,908

(22) PCT Filed: May 26, 2006
(86) PCT No.: PCT/US2006/020580

§ 371 Date: Nov. 6, 2009

Related U.S. Application Data
(60) Provisional application No. 60/684,686, filed on May 26, 2005.
(60) Provisional application No. 60/684,903, filed on May 26, 2005.

(51) Int. Cl. C12Q 1/68

(52) U.S. Cl. 435/6

(57) ABSTRACT

The present invention provides methods of identifying mutations in nucleic acid. Also provided herein are methods of identifying subjects having Hirschsprung disease risk and diagnostic markers for Hirschsprung disease.

### RELATED APPLICATIONS

This application claims the benefit of U.S. Provisional Application Nos. 60/684,686 and 60/684,903, filed May 26, 2005, the entire contents of which are expressly incorporated herein by reference.

### GOVERNMENT SUPPORT

The following invention was supported at least in part by the NIH. Accordingly, the government may have certain rights in the invention.

### BACKGROUND

The identification of common variants that contribute to the genesis of human inherited disorders remains a significant challenge. For example, Hirschsprung disease (HSCR) is a multifactorial, non-Mendelian disorder in which rare high penetrance coding sequence mutations in the receptor tyrosine kinase RET contribute to risk in combination with mutations at other genes.

Hirschsprung disease (HSCR), or congenital aganglionosis with megacolon, occurs in 1 in 5,000 live births. Heritability of HSCR is nearly 100% with clear multigenic inheritance. While RET represents the major implicated HSCR gene^{1,2}, mutations also occur in seven other genes involved in enteric development, specifically ECE1, EDN3, EDNRB, GDNF, NRTN, SOX10, and ZFHX1B². Less than 30% of patients, however, have mutations in these eight genes; thus, additional HSCR-causing mutations in RET and/or at other genes must exist.

Thus, there is a need in the art for methods of identifying variants that contribute to diseases, for example HSCR.

### SUMMARY

Provided herein, in part by using a combination of human genetic, comparative genomic, functional, and population genetic analyses, are methods of identifying mutations in nucleic acid, and specifically methods of identifying subjects having Hirschsprung disease risk.

We have used family-based association studies to identify a disease interval, and integrated this with comparative and functional genomic analysis to prioritize conserved and functional elements within which mutations can be sought. We now show that a common, non-coding RET variant within a conserved enhancer-like sequence in intron 1 is significantly associated with HSCR susceptibility and makes 20-fold greater contribution to risk than do rare alleles. This mutation reduces in vitro enhancer activity markedly, has low penetrance, has different genetic effects in males and females, and explains several features of the complex inheritance pattern of HSCR. Thus, common, low penetrance variants, identified by association studies, can underlie both common and rare diseases.

In one aspect, provided herein are methods of identifying a mutation in DNA, comprising predicting a genetic interval for a disease; comparing orthologous sequences to refine a putative functional interval; and sequencing the putative functional interval subjects to identify mutations.

In one aspect, provided herein are methods of identifying a mutation in DNA, comprising predicting a genetic interval harboring mutations that contribute to disease susceptibility; comparing orthologous sequences to refine a putative functional interval; and sequencing the putative functional interval subjects to identify mutations.

In one embodiment, the methods further comprise classifying the refined interval into one or more of coding, non-coding, functional and non-functional sequences.

In one related embodiment, the further comparing is after comparing orthologous sequences.

In one embodiment, the predicting comprises one or more of transmission disequilibrium tests (TNTs), linkage, or association studies.

In another embodiment, the subjects comprise individuals from affected families.

In one embodiment, the subjects comprise affected and unaffected individuals.

In another embodiment, mutations are over-represented in affected subjects as compared to normal subjects.

In another embodiment, the mutation is associated with a multigenic disease.

In one embodiment, the multigenic disease comprise one or more of mental illness, cancer, cardiovascular disease, congenital anomalies, metabolic disorder inc but not limited to diabetes, susceptibility to infection, drug response, or drug tolerance.

In one embodiment, the mutation comprises a variant of RET.

In one related embodiment, the RET variant comprises RET+3:T.

In another embodiment, the mutations are one or more of associated with a disease susceptibility, are causative of disease, are contributory to disease,

In one embodiment, the mutation comprises a single nucleotide polymorphism, a multi-nucleotide polymorphism, an insertion, a deletion, a repeat expansion, genomic rearrangements, or segmental amplification.

In another embodiment, the orthologous sequences comprise vertebrate sequences.

In one embodiment, the vertebrate sequences comprise mammalian, reptilian, avian, amphibians, or osteichthyes.

In one embodiment, at least two orthologous sequences are compared to refine the interval.

In one embodiment, the interval is refined by at least 20 fold.

In one related embodiment, the interval is refined by about 10 fold.

In another related embodiment, the interval is refined by about 5 fold.

In one aspect, provided herein are methods of identifying a diagnostic marker for a disease, comprising predicting a genetic interval for a disease; comparing orthologous sequences to refine the interval; and sequencing the refined interval in affected and unaffected subjects to thereby identify a diagnostic marker associated with disease susceptibility, wherein the marker is over represented in affected subjects compared to unaffected subjects.

In one embodiment, the methods further comprise classifying the refined interval into one or more of coding, non-coding, functional and non-functional sequences.

In one embodiment, the further comparing is after comparing orthologous sequences.

In another embodiment, the predicting comprises one or more of transmission disequilibrium tests (TDTs), linkage, or association studies.

In one embodiment, the subjects comprise affected and unaffected individuals.

In another embodiment, mutations are over-represented in affected subjects as compared to normal subjects.

In one embodiment, the mutation is associated with a multigenic disease.

In another embodiment, the multigenic disease comprise one or more of mental illness, cancer, cardiovascular disease, congenital anomalies, metabolic disorder inc but not limited to diabetes, susceptibility to infection, drug response, or drug tolerance.

In another embodiment, the mutations are one or more of associated with a disease susceptibility, are causative of disease, are contributory to disease,

In one embodiment, mutation comprises a single nucleotide polymorphism, a multi-nucleotide polymorphism, an insertion, a deletion, a repeat expansion, genomic rearrangements, or segmental amplification.

In one embodiment, the orthologous sequences comprise vertebrate sequences.

In another embodiment, the vertebrate sequences comprise mammalian, reptilian, avian, amphibians, or osteichthyes.

In one embodiment, at least two orthologous sequences are compared to refine the interval.

In one related embodiment, the interval is refined by at least 20 fold.

In another related embodiment, the interval is refined by about 10 fold.

In yet another related embodiment, the interval is refined by about 5 fold.

In one embodiment, the methods may further comprise characterizing the marker. In one embodiment, characterizing comprises one or more of expression analysis, promoter analysis, regulatory element analysis, knock-out analysis, or knock-down analysis. Methods of analysis are well known to one of skill in the art. In a related embodiment, one or more of the analyses are done with a transgenic animal or a cell line.

According to one aspect, provided herein are methods of identifying a subject having Hirschsprung disease risk comprising detecting in the subject a mutation in the receptor tyrosine kinase RET, wherein the RET+3:T allele is associated with disease risk.

In one embodiment, RET is a maker for segmental forms of HSCR.

In one embodiment, the subject is a member of an affected family.

### BRIEF DESCRIPTION OF THE DRAWINGS

FIG. 1 depicts transmission disequilibrium tests (TDT). (a) TDT tests of individual SNPs. The region of 10q11.21 including RET, GALNACT-2, RASGEFLA. Horizontal line at 50% transmission indicates expectation under the null hypothesis. The * identifies RET+3. Exons are marked by coloured boxes. Black rectangle represents the 27-kb area displayed in FIG. 3a. (b) Exhaustive Allelic TDT (EATDT). The most 5′ SNP shown is RET-5, the most 3′ SNP is X2EagI. Counts of transmitted and untransmitted chromosomes are given in columns to the right. All haplotypes with permutation-based p values less than or equal to the single most significantly associated SNP (RET+3) are shown.

FIG. 2 depicts the identification and characterization of conserved sequence elements within 350 kb encompassing RET. (a) Multi-PIP alignment of genomic sequence from 12 vertebrates compared to the human. Red: greater than 75% sequence identity over 100 nucleotides; green: greater than 50% sequence identity over 100 nucleotides, blue: gaps in contig of 500 nucleotides or more. (b) Northern blots showing expression of GALNACT-2 (GN2) and RASGEF1A (RG1A) in adult mouse tissues. (c) and (d) Expression of RET, GALNACT-2 (GN2) and RASGEF1A (RG1A) by RT-PCR in (c) embryonic mouse and (d) adult human tissues.

FIG. 3 depicts (a) VISTA plot displaying percent identity between mouse and human in the 5′ region of RET. Estimated transmission frequencies to affected offspring are shown by red circles. (b) Reporter gene expression in Neuro-2a cells using amplicons MCS+9.7 and MCS+5.1/9.7 (Mutant and wild type correspond to nucleotides T and C, respectively). The smaller of the tested constructs (MCS+9.7 only) is bracketed in red. The MCS+5.1/9.7 amplicon encompassing both MCS+9.7 and the adjacent MCS+5.1 is bracketed in green. All assays were conducted in triplicate and were repeated three times (9 data points total); error bars represent standard error.

FIG. 4 depicts worldwide allele frequencies of RET+3. Frequencies of the putative wild type (green, C) and mutant (yellow, T) alleles are given for 51 populations comprising 1,064 individuals from the CEPH Human Genome Diversity Panel.

FIG. 5 depicts nucleotide alignment of multiple mammalian sequences showing the complete sequence of MCS+9.7. Additional sequence flanking the MCS is shown in lower-case, gray lettering. Position of the functional SNP RET+3 is highlighted in red.

### DETAILED DESCRIPTION

Provided herein are methods relating to identifying diagnostic markers, identifying mutations in DNA and identifying subjects having Hirschspring disease risk. In particular, we have shown methods comprising comparing an identified genetic interval to orthologous sequences refines the interval.

In part, the invention is based on the use of family-based association studies to identify a disease interval, and integrated this with comparative and functional genomic analysis to prioritize conserved and functional elements within which mutations can be sought. For example, a common, non-coding RET variant within a conserved enhancer-like sequence in intron 1 is significantly associated with HSCR susceptibility and makes 20-fold greater contribution to risk than do rare alleles. This mutation reduces in vitro enhancer activity markedly, has low penetrance, has different genetic effects in males and females, and explains several features of the complex inheritance pattern of HSCR. Thus, common, low penetrance variants, identified by association studies, can underlie both common and rare diseases.

“Mutation,” as used herein, refers, for example, to a polymorphism or marker that occurs in those at risk of developing a disease, is associated with a disease or causative of a disease. In certain instances, the mutation may be strongly correlated with the presence of a particular disorder (e.g., the presence of such mutation indicating a high risk of the subject being afflicted with a disease). However, “mutation” as used herein can also refer to a specific site and type of polymorphism or marker, without reference to the degree of risk that particular mutation poses to an individual for a particular disease. Mutations, as used herein, are over-represented in affected subjects as compared to normal subjects and may be associated with a multigenic disease. The multigenic disease may comprise, for example, one or more of mental illness, cancer, cardiovascular disease, congenital anomalies, metabolic disorder inc but not limited to diabetes, susceptibility to infection, drug response, or drug tolerance. The mutation may comprises a variant of RET, for example, the RET variant RET+3:T. Mutations may be one or more of associated with a disease susceptibility, causative of disease, or contributory to disease and the like. Mutations, as used herein may comprises a single nucleotide polymorphism, a multi-nucleotide polymorphism, an insertion, a deletion, a repeat expansion, genomic rearrangements, or segmental amplification.

“Linked,” as used herein, refers, for example, to a region of a chromosome shared more frequently in family members affected by a particular disease than would be expected by chance, thereby indicating that the gene or genes within the linked chromosome region contain or are associated with a marker or polymorphism that is correlated to the presence of, or risk of, disease. Once linkage is established, for example, by association studies (linkage disequilibrium) can be used to narrow the region of interest or to identify the risk-conferring gene associated with a disease.

“Associated with” when used to refer for example to a marker or polymorphism and a particular gene means that the polymorphism or marker is either within the indicated gene, or in a different physically adjacent gene on that chromosome. In general, such a physically adjacent gene is on the same chromosome and within 2, 3, 5, 10 or 15 centimorgans of the named gene (i.e., within about 1 or 2 million base pairs of the named gene). The adjacent gene may span over 5, 10 or even 15 megabases. Polymorphisms may be functional polymorphisms. “Associated with,” in reference to a mutation being associated with a disease, refers to, for example, a statistical association.

A “centimorgan” as used herein refers to a unit of measure of recombination frequency. One centimorgan is equal to a 1% chance that a marker at one genetic locus will be separated from a marker at a second locus due to crossing over in a single generation. In humans, one centimorgan is equivalent, on average, to one million base pairs.

Markers and polymorphisms of this invention (e.g., genetic markers such as single nucleotide polymorphisms, restriction fragment length polymorphisms and simple sequence length polymorphisms) can be detected directly or indirectly. A marker can, for example, be detected indirectly by detecting or screening for another marker that is tightly linked (e.g., is located within 2 or 3 centimorgans) of that marker. Additionally, the adjacent gene can be found within an approximately 15 cM linkage region surrounding the chromosome, thus spanning over 5, 10 or even 15 megabases.

The presence of a marker or polymorphism associated with a gene linked to, for example, a disease, for example Hirschsprung disease, indicates that the subject is afflicted with the disease or is at risk of developing the disease and/or is at risk of developing the disease. A subject who is “at increased risk of developing a disease” is one who is predisposed to the disease, has genetic susceptibility for the disease and/or is more likely to develop the disease than subjects in which the detected polymorphism is absent. A subject who is “at increased risk of developing a disease at an early age” is one who is predisposed to the disease, has genetic susceptibility for the disease and/or is more likely to develop the disease at an age that is earlier than the age of onset in subjects in which the detected polymorphism is absent. Thus, the marker or polymorphism can also indicate “age of onset” of a disease. The methods described herein can be employed to screen for any type of disease, including, for example, multigenic diseases, mental illness, cancer, cardiovascular disease, congenital anomalies, metabolic disorder inc but not limited to diabetes, susceptibility to infection, drug response, or drug tolerance, and the like.

Subjects, include, for example, mammals and specifically human subjects, including male and female subjects of any age or race. Suitable subjects include, but are not limited to, those who have not previously been diagnosed with a disease, those who have previously been determined to be at risk of developing a disease and/or at risk of developing a disease at an early age, and those who have been initially diagnosed with a disease or who are suspected of having a disease where confirming and/or prognostic information is desired. Thus, it is contemplated that the methods described herein can be used in conjunction with other clinical diagnostic information known or described in the art used in the evaluation of subjects with a disease or suspected to be at risk for developing such disease. Subjects may also comprise individuals from affected families and individuals from unaffected families.

The present invention discloses methods of screening a subject for Hirschsprung disease. The method comprises the steps of: detecting the presence or absence of a marker for Hirschsprung disease, and/or a polymorphism associated with a gene linked to Hirschsprung disease, with the presence of such a marker or polymorphism indicating that subject has the disease, and/or is at increased risk of developing Hirschsprung disease.

The detecting step can include determining whether the subject is heterozygous or homozygous for the marker and/or polymorphism, with subjects who are at least heterozygous for the polymorphism or marker being at increased risk for a disease. The step of detecting the presence or absence of the marker or polymorphism can include the step of detecting the presence or absence of the marker or polymorphism in both chromosomes of the subject (i.e., detecting the presence or absence of one or two alleles containing the marker or polymorphism). More than one copy of a marker or polymorphism (i.e., subjects homozygous for the polymorphism) can indicate a greater risk of developing a disease.

The detecting step can be carried out in accordance with known techniques (See, e.g., U.S. Pat. Nos. 6,027,896 and 5,508,167 to Roses et al.), such as by collecting a biological sample containing nucleic acid (e.g., DNA) from the subject, and then determining the presence or absence of nucleic acid encoding or indicative of the polymorphism or marker in the biological sample. Any biological sample that contains the nucleic acid of that subject can be employed, including tissue samples and blood samples, with blood cells being a particularly convenient source.

Determining the presence or absence of a particular polymorphism or marker can be carried out, for example, with an oligonucleotide probe labeled with a suitable detectable group, and/or by means of an amplification reaction (e.g., with oligonucleotide primers) such as a polymerase chain reaction (PCR) or ligase chain reaction (the product of which amplification reaction can then be detected with a labeled oligonucleotide probe or a number of other techniques). Further, the detecting step can include the step of determining whether the subject is heterozygous or homozygous for the particular polymorphism or marker, as described herein. Numerous different oligonucleotide probe assay formats are known which can be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (the entire contents of each of which are incorporated herein by reference). The oligonucleotides can be used to hybridize to the nucleic acids of this invention. In some embodiments, the oligonucleotides can be from 2 to 100 nucleotides and in other embodiments, the oligonucleotides can be 5, 10, 12, 15, 18, 20, 25, 30 35, 40 45 or 50 bases, including any value between 5 and 50 not specifically recited herein (e.g., 16 bases; 34 bases). Determining the presence or absence of a particular polymorphism may also be carried out by sequencing the relevant nucleic acid.

Amplification of a selected, or target, nucleic acid sequence can be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Q.beta. replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra).

As used here, “predicting a genetic interval for a disease,” refers to, for example, identifying an interval associated with a disease using for example, one or more genetic tests, e.g., of transmission disequilibrium tests (TNTs), linkage, or association studies.

As used here, “comparing orthologous sequences to refine a putative functional interval,” refers to, for example the use of at least one orthologous sequence to the interval. The orthologous sequence refines the interval, by, for example, revealing the evolutionarily conserved regions of the interval that are more likely to be under selective pressure. Thus, differences or mutations found in these regions are more likely to be associated with disease. One or more orthologous sequences may be compared to the interval for further refining. The comparing can be done by software, hardware or by an individual, for example by methods described infra in the Examples. Orthologous sequences comprise, for example, vertebrate sequences. Orthologous sequences may also be from single celled organisms, e.g., yeast, bacteria, viruses, and the like. Vertebrate sequences comprise, for example, mammalian, reptilian, avian, amphibians, or osteichthyes, and the like.

As used here, “a putative functional interval,” refers to, for example, to an interval shown to be associated by, for example by genetic studies, including, transmission disequilibrium tests (TNTs), linkage, or association studies. These methods are useful in predicting the interval.

Sequencing the putative functional interval subjects to identify mutations can be by any known or future developed sequencing methods.

In one embodiment, further comparing is after comparing orthologous sequences.

In one embodiment, one orthlogous sequence is compared to refine the interval. In another embodiment, at least two orthologous sequences are compared to refine the interval. In one embodiment, the interval is refined by the comparison to one or more orthologous sequences by at least about 50 fold, at least about 40 fold, at least about 30 fold, at least about 25 fold, at least about 20 fold, at least about 15 fold, by at least about 10 fold, or at least about 5 fold.

“Classifying the refined interval,” as used herein refers to, for example, defining function or type of sequence that makes up the interval. The classifications include, for example, one or more of coding, non-coding, functional and non-functional sequences. Non-coding sequences may also be classified as functional sequences.

Methods of predicting an interval comprise, for example, multi-analytical approaches including both parametric lod score and non-parametric affected relative pair methods. Maximized parametric lod scores (MLOD) for each marker may be calculated, for example, by using VITESSE and HOMOG program packages (O'Connell & Weeks, Nat. Genet. 11:402 (1995); Ott, Analysis of Human Genetic Linkage. (The Johns Hopkins University Press, Baltimore, Ed. 3, 1999); The MLOD is the lod score maximized over the two genetic models tested, allowing for genetic heterogeneity. Dominant and recessive low-penetrance (affecteds-only) models may be considered. Methods may be further based on prevalence estimates and for example, age-dependent or incomplete penetrance. Disease allele frequencies of 0.001 for the dominant model and 0.20 for the recessive model may be used. Marker allele frequencies may be generated, for example, from related or unrelated individuals. Multipoint non-parametric lod scores (LOD*) may be calculated, for example, using GENEHUNTER-PLUS software (Kong & Cox, Am. J. Hum. Genet. 61:1179 (1997)) and sex-averaged intermarker distances. In contrast to non-parametric linkage approaches which consider allele sharing in pairs of affected siblings [Risch, Am. J. Hum. Genet. 46:222 (1990)], GENEHUNTER-PLUS considers allele sharing across pairs of affected relatives (or all affected relatives in a family) in moderately sized pedigrees.

Depending upon the disease being studied and due to the potential genetic heterogeneity in this sample, samples may stratified, or example by age of onset.

In one embodiment, an initial complete genomic screen is used to identify regions of the genome likely harboring susceptibility loci for more thorough analysis. Genetic heterogeneity likely reduces the power to detect statistically significant evidence of linkage using the traditional criterion, lod scores of from about 3 to about 1 may be used in the overall sample for consideration of a region as interesting and warranting initial follow-up. Regions may be prioritized into two groups: regions generating lod scores >1 on both two-point and multipoint analyses and while regions with lod scores >1. While this approach may increase the number of false-positive results that are examined in more detail, it decreases the more serious (in this case) false-negative rate.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to a disease; or to refer to an individual's response to a drug; or to refer to symptoms of, or susceptibility to side effects to a drug. In addition, the terms “trait” or “phenotype” may be used herein to refer to symptoms of, or susceptibility to a disease involving arachidonic acid metabolism; or to refer to an individual's response to an agent acting on arachidonic acid metabolism; or to refer to symptoms of, or susceptibility to side effects to an agent acting on arachidonic acid metabolism.

The term “allele” is used herein to refer to variants of a nucleotide sequence. A biallelic polymorphism has two forms. Typically the first identified allele is designated as the original allele whereas other alleles are designated as alternative alleles. Diploid organisms may be homozygous or heterozygous for an allelic form.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker consists of determining the specific allele or the specific nucleotide carried by an individual at a biallelic marker.

The term “haplotype” refers to one or more alleles present on the same chromosome in an individual or a sample. In the context of the present invention a haplotype preferably refers to a combination of biallelic marker alleles found in a given individual and which may be associated with a phenotype.

The term “polymorphism” as used herein refer to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site may be occupied by two different nucleotides.

The terms “biallelic polymorphism” and “biallelic marker” are used interchangeably herein to refer to a polymorphism having two alleles at a fairly high frequency in the population, preferably a single nucleotide polymorphism. A “biallelic marker allele” refers to the nucleotide variants present at a biallelic marker site. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker.”

The term “upstream” is used herein to refer to a location which, is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

A “promoter” refers to a DNA sequence recognized by the synthetic machinery of the cell required to initiate the specific transcription of a gene.

A sequence which is “operably linked” to a regulatory sequence such as a promoter means that said regulatory element is in the correct location and orientation in relation to the nucleic acid to control RNA polymerase initiation and expression of the nucleic acid of interest.

As used herein, the term “operably linked” refers to a linkage of polynucleotide elements in a functional relationship. For instance, a promoter or enhancer is operably linked to a coding sequence if it affects the transcription of the coding sequence. More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide.

The TDT (Spielman et al. (1993) Am J Hum Genet. 52: 506-16) is a test for both association and for linkage, more specifically, it tests for linkage in the presence of association. Thus, if association does not exist at the locus of interest, linkage will not be detected even if it exists. It is for this reason that the test has been included in this section. It may be used as an initial test, but is more commonly used when tentative evidence for association has already been identified. In this case, a positive result will not only confirm the initial association, but also provide evidence for linkage.

Multi-allele Transmission Disequilibrium Test (TDT). TDT is at widely used method for family-based genetic study (Spielman et al., Transmission test for linkage disequilibrium: the insulin gene region and insulin-dependent diabetes mellitus (IDDM), Am. J. Hum. Genet., 1993 March; 52 (3):506-16), where parents and children in a family are typed. Testing for linkage in the presence of linkage disequilibrium (association), TDT can be very powerful to identify susceptibility locus, especially when the effect is small, as is often the case with complex genetic trait. Although the original TDT test was developed to analyze biallelic markers, new statistics have been developed to accommodate the availability of multiallelic markers or haplotypes (Spielman et al., The TDT and other family-based tests for linkage disequilibrium and association, Am. J. Hum. Gent., 1996 November; 59 (5):983-9; Curtis and Sham, Model-free linkage analysis using likelihoods, Am. J. Hum. Genet., 1995 September; 57(3):703-16; Bickeboller et al., Statistical properties of the allelic and genotypic transmission/disequilibrium test for multiallelic markers, Genet. Epidemiol., 1995; 12(6):865-70). Based on survey performed by Kaplan (Kaplan et al., Power studies for the transmission/disequilibrium tests with multiple alleles, Am. J. Hum. Genet., 1997 March; 60(3):691-702) on those methods, we have chosen the marginal statistics with only heterozygous parents (T.sub.mhet) by Spielman and Ewens (Spielman et al., The TDT and other family-based tests for linkage disequilibrium and association, Am. J. Hum. Genet., 1996 November; 59(5):983-9), because it has equivalent power to the other multi-allelic tests and gives a valid chi-square test of linkage. Multi-allele TDT can be readily applied to patterns because of the multi-allele or multi-genotype nature of a pattern. In a TDT test on a pattern, each observed permutation of a pattern is treated as column and row headings in a TDT contingency table. Corresponding chi-square value is calculated based on described (Spielman et al., The TDT and other family-based tests for linkage disequilibrum and association, Am. J. Hum. Genet., 1996 November; 59 (5):983-9) and P value is assigned according to default or reference distribution simulated by Monte Carlo. This statistics can only be applied to patterns identified in a family-based association study design.

The Quantitative Transmission Disequilibrium Test (OTDT) Analysis was proposed by George et al. [1999] was used to conduct QTDT analysis. This test detects linkage in the presence of association. This test detects linkage in the presence of association. The maximum likelihood estimates of the parameters and the standard errors of the estimates are computed by numerical methods. These procedures are implemented in the program ASSOC of the S.A.G.E. [1998] software package.

Single permutation tests have been used in mapping studies before (Churchill and Doerge 1994, Laitinen et al. 1997, Long and Langley 1999). However, if more complex data is to be analyzed, these single permutation tests are too expensive and computationally very ineffective and even inoperative.

Haplotype-based Haplotype Relative Risk (HHRR). HHRR test is another method for family-based studies (Terwilliger et al., A haplotype-based ‘haplotype relative risk’ approach to detecting allelic associations, Hum. Hered., 1992; 42(6):337-46, 1992). It is a variation of the Haplotype Relative Risk (HRR) method, which is genotype-based. In Rubinstein's Genotype-based haplotype relative risk (GHRR) method, the affected children's genotypes at a marker locus are used as cases and artificial genotypes made up of the alleles not transmitted to the children from their parents are used as controls. For each haplotype of interest, a 2×2 contingency table is constructed and used to record the number of cases and controls with or without that haplotype. In contrast, HHRR utilizes haplotypes rather than genotypes. In particular, transmitted chromosomes are treated as cases and untransmitted chromosomes are used as controls, A 2×2 table is constructed the same as for GHRR. HHRR can be extended to be applied to patterns because of the similarity between a pattern and a multi-marker haplotype. In a HHRR test for a pattern, the observed counts for the pattern in cases and in controls and the observed counts for all other permutations on markers in that pattern in cases and contros are recorded in the 2×2 contingency table. Upon the calculation of chi-square values, P values are assigned according to default distribution or reference distribution simulated by Monte Carlo.

Statistical significant based on uncorrelated pattern formation (Califano et al., Analysis of gene expression microarrays for phenotype classification, Proc. Int. Conf. Intell. Syst. Mol. Biol., 2000; 8:75-85).

In another aspect, it will be understood that the invention provides systems that may be employed to compare the orthologous sequences. The systems may be machines as well as software tools and can include devices for processing sequence data as well as data visualization tools which can highlight patterns in data that is visually displayed. The system may comprise a conventional data processing platform such as an IBM PC-compatible computer running the Windows operating systems, or a SUN workstation running a Unix operating system. Alternatively, the system can comprise a dedicated processing system that includes an embedded programmable data processing system. For example, the system can comprise a single board computer system that has been integrated into a system for sequencing genomic data, identifying SNPs or markers, collecting expression data, or for performing other laboratory processes. The system may also be able to process classifying the sequence data into one or more of coding, non-coding, functional and non-functional sequences.

As used herein, the term “genome” is intended to mean the full complement of chromosomal DNA found within the nucleus of a eukaryotic cell. The term can also be used to refer to the entire genetic complement of a prokaryote, virus, mitochondrion or chloroplast or to the haploid nuclear genetic complement of a eukaryotic species.

As used herein, the term “genomic DNA” or “gDNA” is intended to mean one or more chromosomal polymeric deoxyribonucleotide molecules occurring naturally in the nucleus of a eukaryotic cell or in a prokaryote, virus, mitochondrion or chloroplast and containing sequences that are naturally transcribed into RNA as well as sequences that are not naturally transcribed into RNA by the cell. A gDNA of a eukaryotic cell contains at least one centromere, two telomeres, one origin of replication, and one sequence that is not transcribed into RNA by the eukaryotic cell including, for example, an intron or transcription promoter. A gDNA of a prokaryotic cell contains at least one origin of replication and one sequence that is not transcribed into RNA by the prokaryotic cell including, for example, a transcription promoter. A eukaryotic genomic DNA can be distinguished from prokaryotic, viral or organellar genomic DNA, for example, according to the presence of introns in eukaryotic genomic DNA and absence of introns in the gDNA of the others.

As used herein, the term “detecting” is intended to mean any method of determining the presence of a particular molecule such as a nucleic acid having a specific nucleotide sequence. Techniques used to detect a nucleic acid include, for example, hybridization to the sequence to be detected. However, particular embodiments of this invention need not require hybridization directly to the sequence to be detected, but rather the hybridization can occur near the sequence to be detected, or adjacent to the sequence to be detected. Use of the term “near” is meant to imply within about 150 bases from the sequence to be detected. Other distances along a nucleic acid that are within about 150 bases and therefore near include, for example, about 100, 50 40, 30, 20, 19, 18, 17, 16, 15, 14, 13, 12, 11, 10, 9, 8, 7, 6, 5, 4, 3, 2, or 1 bases from the sequence to be detected. Hybridization can occur at sequences that are further distances from a locus or sequence to be detected including, for example, a distance of about 250 bases, 500 bases, 1 kilobase or more up to and including the length of the target nucleic acids or genome fragments being detected.

Examples of reagents which are useful for detection include, but are not limited to, radiolabeled probes, fluorophore-labeled probes, quantum dot-labeled probes, chromophore-labeled probes, enzyme-labeled probes, affinity ligand-labeled probes, electromagnetic spin labeled probes, heavy atom labeled probes, probes labeled with nanoparticle light scattering labels or other nanoparticles or spherical shells, and probes labeled with any other signal generating label known to those of skill in the art. Non-limiting examples of label moieties useful for detection in the invention include, without limitation, suitable enzymes such as horseradish peroxidase, alkaline phosphatase, .beta.-galactosidase, or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6.sup.th Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S or ³H.

Mutation is meant to encompass single nucleotide polymorphisms (SNPs), mutations, variable number of tandem repeats (VNTRs) and single tandem repeats (STRs), other polymorphisms, insertions, deletions, splice variants or any other known genetic markers. Exemplary resources that provide known SNPs and other genetic variations include, but are not limited to, the dbSNP administered by the NCBI and available online at ncbi.nlm.nih.gov/SNP/ and the HCVBASE database described in Fredman et al. Nucleic Acids Research, 30:387-91, (2002) and available online at hgvbase.cgb.ki.se/.

As used herein, the term “corresponding to,” when used in reference to a locus, is intended to mean having a nucleotide sequence that is identical or complimentary to the sequence of the locus, or a diagnostic portion thereof. Exemplary diagnostic portions include, for example, nucleic acid sequences adjacent or near to the locus of interest.

As used herein, the term “multiplex” is intended to mean simultaneously conducting a plurality of assays on one or more sample. Multiplexing can further include simultaneously conducting a plurality of assays in each of a plurality of separate samples. For example, the number of reaction mixtures analyzed can be based on the number of wells in a multi-well plate (or holes in a through-hole array) and the number of assays conducted in each well can be based on the number of probes that contact the contents of each well. Thus, 96 well, 384 well or 1536 well microtiter plates will utilize composite arrays comprising 96, 384 and 1536 individual arrays, although as will be appreciated by those in the art, not each microtiter well need contain an individual array. Depending on the size of the microtiter plate and the size of the individual array, very high numbers of assays can be run simultaneously; for example, using individual arrays of 2,000 and a 96 well microtiter plate, 192,000 experiments can be done at once; the same arrays in a 384 microtiter plate yields 768,000 simultaneous experiments, and a 1536 microtiter plate gives 3,072,000 experiments. Although multiplexing has been exemplified with respect to microtiter plates, it will be understood that other formats can be used for multiplexing including, for example, those described in U.S. 2002/0102578 A1.

### Predictive Medicine

The present invention is based at least in part, on the identification of alleles that are associated (to a statistically significant extent) with the development of a Hirschsprung disease in subjects. Therefore, detection of these alleles, alone or in conjunction with another means in a subject indicate that the subject has or is predisposed to the development of a Hirschsprung disease. For example, polymorphic alleles which are associated with a propensity for developing Hirschsprung disease as described herein or an allele that is in linkage disequilibrium with one of the aforementioned alleles. In a preferred embodiment, this allelic pattern permits the diagnosis of a Hirschsprung disease disorder

Detection of the RET+3 allelic variant in an individual suggests an increased likelihood of developing Hirschsprung disease in comparison to a control individual who does not carry the allele variant. However, because these alleles are in linkage disequilibrium with other alleles, the detection of such other linked alleles can also indicate that the subject has or is predisposed to the development of a Hirschsprung disease. These alleles may be identified by known methods in the art.

One of skill in the art can readily identify other alleles (including polymorphisms and mutations) that are in linkage disequilibrium with an allele associated with a disease. For example, a nucleic acid sample from a first group of subjects without the disease can be collected, as well as DNA from a second group of subjects with the disease. The nucleic acid sample can then be compared to identify those alleles that are over-represented in the second group as compared with the first group, wherein such alleles are presumably associated with the disease. Alternatively, alleles that are in linkage disequilibrium with the disease associated allele can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus, race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state.

Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

Appropriate probes may be designed to hybridize to a specific genes identified by methods described herein. For example, the human genome database collects intragenic SNPs, is searchable by sequence and currently contains approximately 2,700 entries (http://hgbase.interactiva.de). Also available is a human polymorphism database maintained by the Massachusetts Institute of Technology (MIT SNP database (http://www.genome.wi.mit.edu/SNP/human/index.html)). From such sources SNPs as well as other human polymorphisms may be found.

### Detection of Alleles

Many methods are available for detecting mutations. The preferred method for detecting a mutation will depend, in part, upon the molecular nature of the mutation. For example, the various allelic forms of the mutation may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. For example, several include dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127).

In another embodiment of the invention, a solution-based method is used for determining the identity of the nucleotide of a polymorphic site, e.g., mutation. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer. An alternative method, known as Genetic Bit Analysis or GBA™ is described by Goelet, P. et al. (PCT Appln. No. 92/15712). Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1: 159-164 (1992); Ugozzoli, L. et al., GATA 9:107-112 (1992); Nyren, P. et al., Anal. Biochem. 208:171-175 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express an gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele and having about 5, 10, 20, 25, or 30 nucleotides around the mutation or polymorphic region. In a preferred embodiment of the invention, several probes capable of hybridizing specifically to other allelic variants involved in a Hirschsprung disease are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In yet another embodiment, any of a variety of sequencing reactions known in the art can be used to directly sequence the allele. Exemplary sequencing reactions include those based on techniques developed by Maxim and Gilbert ((1977) Proc. Natl. Acad Sci USA 74:560) or Sanger (Sanger et al (1977) Proc. Nat. Acad. Sci. USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out. Single molecule sequencing methods may also be used.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify alocus allele. For example, single strand conformation polymorphism (SSCP) may be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl. Acad. Sci. USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989), Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of an locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for developing a Hirschsprung disease. This kit may contain one or more oligonucleotides, including 5′ and 3′ oligonucleotides that hybridize 5′ and 3′ to at least one allele of an locus haplotype. PCR amplification oligonucleotides should hybridize between 25 and 2500 base pairs apart, preferably between about 100 and about 500 bases apart, in order to produce a PCR product of convenient size for subsequent analysis. Kits may also include sequence reagents and other reagents necessary for the methods described herein.

Exemplary primers for use in the diagnostic methods include RETX10F: 59-TTCCCTGAGGAGGAGAAGTGC-39 and RETX12R: 59-CACTTTTCCAAATTCGCCTT-39. Other exemplary primers may be found, for example, in Minerva M. Carrasquillo et al., “Genome-wide association study and mouse model identify interaction between RET and EDNRB pathways in Hirschsprung disease,” nature genetics, vol. 32 (2002); Stacey Bolk et al., “A human model for multigenic inheritance: Phenotypic expression in Hirschsprung disease requires both the RET gene and a new 9q31 locus,” PNAS, vol. 97, pp 268-273 (2000); and Stacey Bolk Gabriel, et al., “Segregation at three loci explains familial and population risk in Hirschsprung disease,” Nature Genetics, vol 31 (2002).

The design of additional oligonucleotides for use in the amplification and detection of polymorphic alleles by the method of the invention is facilitated by the availability of updated sequence information from human chromosomes. Suitable primers for the detection of a human polymorphism in these genes can be readily designed using sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1.alpha., interleukin-1.beta., and Interleukin-1 Receptor Antagonist Genes” Genomics 19: 382 (1995); Nothwang H. G., et al. “Molecular Cloning of the Interleukin-1 gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project at the URL http://www.gdb.org).

### Therapeutics

Modulators of affected genes or a protein encoded by a gene that is in linkage disequilibrium with an gene with a mutation of the invention gene can comprise any type of compound, including a protein, peptide, peptidomimetic, small molecule, or nucleic acid. Preferred agonists include nucleic acids, proteins or a small molecule. Preferred antagonists, which can be identified, for example, using the assays described herein, include nucleic acids (e.g. single (antisense) or double stranded (triplex) DNA or PNA and ribozymes), protein (e.g. antibodies) and small molecules that act to modulate, upregulate, suppress or inhibit transcription and/or protein activity.

### Effective Dose

Toxicity and therapeutic efficacy of such compounds can be determined by standard pharmaceutical procedures in cell cultures or experimental animals, e.g., for determining The LD₅₀ (the dose lethal to 50% of the population) and the E₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissues in order to minimize potential damage to uninfected cells and, thereby, reduce side effects.

Data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulation and Use

Compositions for use in accordance with the present invention may be formulated in a conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the compositions may take the form of, for example, tablets or capsules prepared by conventional means with pharmaceutically acceptable excipients such as binding agents (e.g., pregelatinised maize starch, polyvinylpyrrolidone or hydroxypropyl compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulation and Use

Compositions for use in accordance with the present invention may be formulated in a conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be transmucosal or transdermal. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Assays to Identify Hirschsprung Disease Therapeutics

Based on the identification of mutations that cause or contribute to the development of Hirschsprung disease, the invention further features cell-based or cell free assays, e.g., for identifying Hirschsprung disease therapeutics. In one embodiment, a cell expressing an receptor, or a receptor for a protein that is encoded by a gene which is in linkage disequilibrium with an gene, on the outer surface of its cellular membrane is incubated in the presence of a test compound alone or in the presence of a test compound and another protein and the interaction between the test compound and the receptor or between the protein (preferably a tagged protein) and the receptor is detected, e.g., by using a microphysiometer (McConnell et al. (1992) Science 257:1906). An interaction between the receptor and either the test compound or the protein is detected by the microphysiometer as a change in the acidification of the medium. This assay system thus provides a means of identifying molecular antagonists which, for example, function by interfering with protein-receptor interactions, as well as molecular agonist which, for example, function by activating a receptor.

Cellular or cell-free assays can also be used to identify compounds which modulate expression of a gene or a gene in linkage disequilibrium therewith, modulate translation of an mRNA, or which modulate the stability of an mRNA or protein. Accordingly, in one embodiment, a cell which is capable of producing protein is incubated with a test compound and the amount of protein produced in the cell medium is measured and compared to that produced from a cell which has not been contacted with the test compound. The specificity of the compound vis a vis the protein can be confirmed by various control analysis, e.g., measuring the expression of one or more control genes. In particular, this assay can be used to determine the efficacy of antisense, ribozyme and triplex compounds.

Cell-free assays can also be used to identify compounds which are capable of interacting with a protein, to thereby modify the activity of the protein. Such a compound can, e.g., modify the structure of a protein thereby effecting its ability to bind to a receptor. In a preferred embodiment, cell-free assays for identifying such compounds consist essentially in a reaction mixture containing a protein and a test compound or a library of test compounds in the presence or absence of a binding partner. A test compound can be, e.g., a derivative of a binding partner, e.g., a biologically inactive target peptide, or a small molecule.

Accordingly, one exemplary screening assay of the present invention includes the steps of contacting a protein or functional fragment thereof with a test compound or library of test compounds and detecting the formation of complexes. For detection purposes, the molecule can be labeled with a specific marker and the test compound or library of test compounds labeled with a different marker. Interaction of a test compound with a protein or fragment thereof can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the protein or functional fragment thereof is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) a protein associated with a disease identified by a method described herein or other protein, (ii) an appropriate receptor, and (iii) a test compound; and (b) detecting interaction of the protein and receptor. A statistically significant change (potentiation or inhibition) in the interaction of the protein and receptor in the presence of the test compound, relative to the interaction in the absence of the test compound, indicates a potential antagonist (inhibitor). The compounds of this assay can be contacted simultaneously. Alternatively, a protein can first be contacted with a test compound for an appropriate amount of time, following which the receptor is added to the reaction mixture. The efficacy of the compound can be assessed by generating dose response curves from data obtained using various concentrations of the test compound. Moreover, a control assay can also be performed to provide a baseline for comparison.

Complex formation between a protein and receptor may be detected by a variety of techniques. Modulation of the formation of complexes can be quantitated using, for example, detectably labeled proteins such as radiolabeled, fluorescently labeled, or enzymatically labeled proteins or receptors, by immunoassay, or by chromatographic detection.

It may be desirable to immobilize either the protein or the receptor to facilitate separation of complexes from uncomplexed forms of one or both of the proteins, as well as to accommodate automation of the assay. Binding of protein and receptor can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase fusion proteins can be adsorbed onto glutathione sepharose beads (Sigma Chemical, St. Louis, Miss.) or glutathione derivatized microtitre plates, which are then combined with the receptor, e.g. an ³⁵S-labeled receptor, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes can be dissociated from the matrix, separated by SDS-PAGE, and the level of protein or receptor found in the bead fraction quantitated from the gel using standard electrophoretic techniques such as described in the appended examples. Other techniques for immobilizing proteins on matrices are also available for use in the subject assay. For instance, either protein or receptor can be immobilized utilizing conjugation of biotin and streptavidin.

Transgenic animals can also be made to identify agonists and antagonists or to confirm the safety and efficacy of a candidate therapeutic. Transgenic animals of the invention can include non-human animals containing a Hirschsprung disease causative mutation under the control of an appropriate endogenous promoter or under the control of a heterologous promoter.

The transgenic animals can also be animals containing a transgene, such as reporter gene, under the control of an appropriate promoter or fragment thereof. These animals are useful, e.g., for identifying drugs that modulate production of a protein, such as by modulating gene expression. Methods for obtaining transgenic non-human animals are well known in the art. In preferred embodiments, the expression of the Hirschsprung disease causative mutation is restricted to specific subsets of cells, tissues or developmental stages utilizing, for example, cis-acting sequences that control expression in the desired pattern. In the present invention, such mosaic expression of a protein can be essential for many forms of lineage analysis and can additionally provide a means to assess the effects of, for example, expression level which might grossly alter development in small patches of tissue within an otherwise normal embryo. Toward this end, tissue-specific regulatory sequences and conditional regulatory sequences can be used to control expression of the mutation in certain spatial patterns. Moreover, temporal patterns of expression can be provided by, for example, conditional recombination systems or prokaryotic transcriptional regulatory sequences. Genetic techniques, which allow for the expression of a mutation can be regulated via site-specific genetic manipulation in vivo, are known to those skilled in the art.

The transgenic animals of the present invention all include within a plurality of their cells a Hirschsprung disease causative mutation transgene of the present invention, which transgene alters the phenotype of the “host cell”. In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation of expression of the causative mutation transgene can be regulated via control of recombinase expression.

Use of the cre/loxP recombinase system to regulate expression of a causative mutation transgene requires the construction of a transgenic animal containing transgenes encoding both the Cre recombinase and the subject protein. Animals containing both the Cre recombinase and the Hirschsprung disease causative mutation transgene can be provided through the construction of “double” transgenic animals. A convenient method for providing such animals is to mate two transgenic animals each containing a transgene.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the transactivating protein, e.g. a recombinase or a prokaryotic protein, is delivered to the tissue and caused to be expressed, such as in a cell-type specific manner. By this method, the transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2.sup.b, H-2.sup.d or H-2.sup.q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter which allows reproducible injection of 1-2 μl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene. Transgenic animals may be made by any known or future developed technique, which would be known to one of skill in the art.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. Further, in such embodiments the sequence will be attached to a transcriptional control element, e.g., a promoter, which preferably allows the expression of the transgene product in a specific type of cell.

Retroviral infection can also be used to introduce the transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

The present invention is further illustrated by the following examples which should not be construed as limiting in any way. The contents of all cited references (including literature references, issued patents, published patent applications as cited throughout this application) are hereby expressly incorporated by reference. The practice of the present invention will employ, unless otherwise indicated, conventional techniques that are within the skill of the art. Such techniques are explained fully in the literature. See, for example, Molecular Cloning A Laboratory Manual, (2nd ed., Sambrook, Fritsch and Maniatis, eds., Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; U.S. Pat. No. 4,683,202; and Nucleic Acid Hybridization (B. D. Hames & S. J. Higgins eds., 1984).

The processes and systems described above can be realized as a software component operating on a conventional data processing system such as a Unix workstation. In that embodiment, the process can be implemented as a C language computer program, or a computer program written in any high level language including C++, Fortran, Java or Basic. Additionally, in an embodiment where microcontrollers or DSPs are employed, the process can be realized as a computer program written in microcode or written in a high level language and compiled down to microcode that can be executed on the platform employed. The development of such systems is known to those of skill in the art, and such techniques are set forth in Digital Signal Processing Applications with the TMS320 Family, Volumes, I, II, and III, Texas Instruments (1990). Additionally, general techniques for high level programming are known, and set forth in, for example, Stephen G. Kochan, Programming in C, Hayden Publishing (1993). It is noted that DSPs are particularly suited for implementing signal processing functions, including preprocessing functions such as image enhancement through adjustments in contrast, edge definition and brightness. Developing code for the DSP and microcontroller systems follows from principles well known in the art.

Those skilled in the art will know or be able to ascertain using no more than routine experimentation, many equivalents to the embodiments and practices described herein. For example, the systems and methods described herein may be employed in other applications including financial applications, engineering applications and other applications that would benefit from having patterns found within a large dataset. Accordingly, it will be understood that the invention is not to be limited to the embodiments disclosed herein, but is to be understood from the following claims, which are to be interpreted as broadly as allowed under the law.

### EXAMPLES

### Family-Based Association Studies

Genome sequence data (http://genome.ucsc.edu: build 35) identifies two additional genes in the 350-kb region surrounding RET. GALNACT-2, a chondroitin N-acetylgalactosaminyltransferase^{9,10}, contains 8 exons spanning 46.8-kb and begins 9-kb from the last RET exon. Thirteen exons encode RASGEFLA, a predicted guanyl-nucleotide exchange factor which spans 72-kb and begins 65-kb 3′ to RET. To genetically refine the association within this locus, we initially genotyped 28 single nucleotide polymorphisms (SNP) spanning 175-kb in 126 HSCR-affected individuals and their parents, ascertained from the general outbred population (Table 1). The genomic interval encompasses RET, GALNACT-2 and RASGEF1A.

TABLE 1
Analysis of disease associations

| | | | | | All affected individuals | | | Male offspring | | | Female offspring | | |
|---|---|---|---|---|---|---|---|---|---|---|---|---|---|
| Gene | Marker | dbSNP ID | A1 | A2 | T | U | r | T | U | r | T | U | r |
| 5′ RET | RET−6 | rs3097585 | G | T | 45 | 43 | 0.51 | 27 | 32 | 0.46 | 18 | 11 | 0.62 |
| | RET−5 | rs2742250 | G | C | 58 | 44 | 0.58 | 41 | 27 | 0.60 | 15 | 17 | 0.47 |
| | RET−4 | rs3028707 | A | G | 45 | 33 | 0.58 | 32 | 19 | 0.63 | 13 | 14 | 0.48 |
| | RET−3 | rs3028720 | T | C | 38 | 29 | 0.57 | 27 | 17 | 0.61 | 11 | 12 | 0.48 |
| | RET−2† | rs7417653 | G | C | 69 | 25 | 0.73** | 47 | 14 | 0.77** | 22 | 12 | 0.65 |
| | RET−1† | rs2505997 | C | T | 57 | 19 | 0.75** | 43 | 10 | 0.81** | 14 | 9 | 0.61 |
| RET int1 | RET+1† | rs2435385 | T | C | 78 | 29 | 0.72** | 53 | 17 | 0.76** | 23 | 12 | 0.68 |
| | RET+2† | re2435384 | A | G | 73 | 27 | 0.73** | 50 | 15 | 0.77** | 23 | 12 | 0.68 |
| | 1.15fct† | rs2435382 | A | C | 100 | 27 | 0.79*** | 72 | 14 | 0.84*** | 28 | 13 | 0.68 |
| | RET+3†‡ | rs2435357 | T | C | 101 | 25 | 0.80*** | 73 | 12 | 0.88*** | 28 | 13 | 0.68 |
| | RET+4† | rs752975 | A | G | 74 | 29 | 0.72** | 51 | 17 | 0.75** | 23 | 12 | 0.68 |
| | INT1.4b† | rs2505535 | G | A | 92 | 28 | 0.77*** | 68 | 14 | 0.89*** | 24 | 14 | 0.63 |
| RET protein-coding region | X2Eagi† | rs1800858 | A | G | 96 | 28 | 0.77*** | 72 | 14 | 0.84*** | 24 | 14 | 0.63 |
| | INT8 | rs3026750 | G | A | 60 | 40 | 0.60* | 42 | 22 | 0.66* | 18 | 18 | 0.50 |
| | X13Taql | rs1800881 | G | T | 59 | 38 | 0.61* | 41 | 21 | 0.68* | 18 | 17 | 0.51 |
| | I18Bbvl | rs2742237 | C | G | 69 | 28 | 0.68* | 41 | 14 | 0.75** | 18 | 14 | 0.50 |
| | I18Styl | rs2742239 | A | G | 52 | 27 | 0.68* | 34 | 12 | 0.74** | 18 | 15 | 0.55 |
| | I19Bsgl | rs2075912 | T | C | 55 | 25 | 0.69* | 37 | 12 | 0.76** | 18 | 13 | 0.58 |
| GALNACT-2 | GN−1 | rs3026787 | G | A | 17 | 14 | 0.55 | 15 | 10 | 0.60 | 2 | 4 | 0.33 |
| | GN+1 | rs948706 | C | T | 59 | 29 | 0.67* | 40 | 13 | 0.75** | 19 | 18 | 0.54 |
| | GN+2 | rs1854383 | A | G | 35 | 16 | 0.70* | 27 | 9 | 0.75** | 8 | 8 | 0.57 |
| | GN+3 | rs2435337 | G | C | 57 | 29 | 0.68* | 39 | 14 | 0.74** | 18 | 15 | 0.55 |
| | GN+4 | rs2505558 | C | T | 63 | 59 | 0.52 | 42 | 39 | 0.52 | 21 | 20 | 0.51 |
| | GN+5 | rs2435384 | G | T | 57 | 38 | 0.59 | 39 | 21 | 0.65 | 18 | 18 | 0.50 |
| | GN+6 | rs2435381 | T | C | 55 | 41 | 0.57 | 37 | 28 | 0.57 | 18 | 13 | 0.58 |
| RASGEF1A | RAS+2 | rs1254958 | T | C | 56 | 27 | 0.67* | 38 | 13 | 0.75** | 18 | 14 | 0.55 |
| | RAS+1 | rs1254965 | T | C | 66 | 27 | 0.67* | 38 | 12 | 0.76** | 18 | 16 | 0.55 |
| | RAS−1 | rs1272142 | G | T | 55 | 41 | 0.57 | 38 | 22 | 0.63* | 17 | 19 | 0.47 |
| | RAS−2 | rs1955356 | A | T | 51 | 39 | 0.57 | 33 | 24 | 0.58 | 18 | 15 | 0.55 |

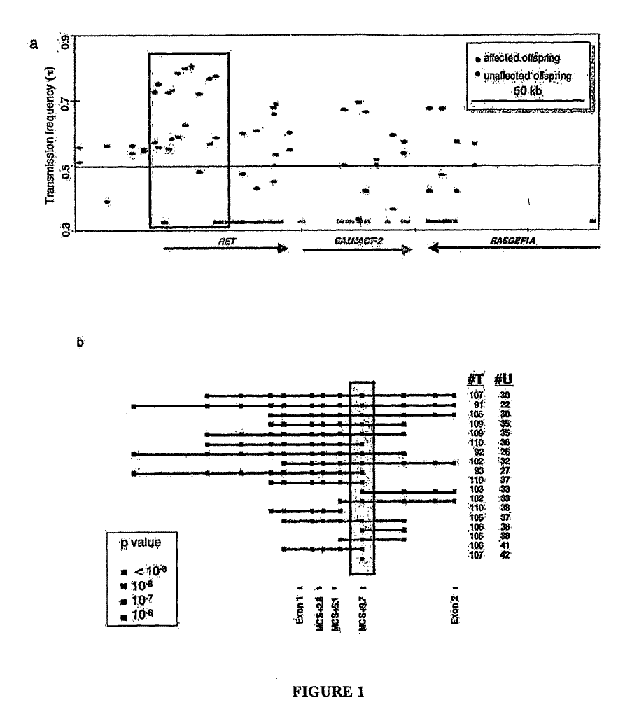

Transmission Disequilibrium Tests (TDT)¹¹ on each SNP demonstrated statistically significant disease associations spanning a region immediately 5′ of RET through RASGEF1A (FIG. 1a; Table 1). Specifically, 13 of 17 RET SNPs, 3 of 7 GALNACT-2 SNPs and 2 of 4 RASGEF1A SNPs tested are significantly associated with HSCR (Table 1), reflecting the high background linkage disequilibrium (LD) in this region (data not shown). However, the greatest statistical significance, and more importantly, the largest transmission distortions (.τ [symbol] 7), occurred among 8 SNPs in a 27.6-kb segment from 4.2-kb 5′ of RET through RET exon 2; (FIG. 1a). Within this region the highest association was within RET intron 1.

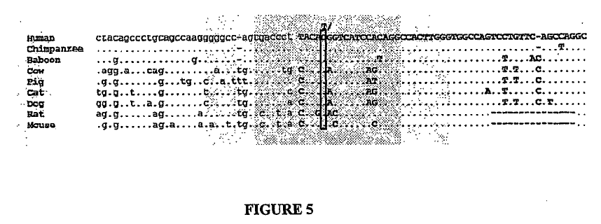

Three re-sequencing experiments were performed and analyzed to identify additional variants, with particular emphasis given to multi-species conserved sequences (MCS; see later) within the 27.6-kb region of highest association. Specifically, we identified the SNP RET+3 (marked by * in FIG. 1a) within MCS+9.7 by re-sequencing HSCR patients from families with demonstrated RET-linkage but no identified coding sequence mutations. TDT of RET+3 in all 126 trios, demonstrated the largest transmission distortion (τ [symbol] 0.8) and the highest statistical significance (p=10⁻¹¹). Interestingly, when association tests are factored by offspring gender, a known risk factor in HSCR, RET+3 and the adjacent marker 1.1SfcI (3.3 kb away) are the only two SNPs demonstrating association in females. Two additional variants (rs2506005, rs2506004) lie within MCS+9.7 which are located 76 nt 5′ and 217 nt 3′ of RET+3, respectively; both are in complete linkage disequilibrium with RET+3 and each other. The HSCR-associated allele at each of these additional SNPs is the ancestral allele. Interestingly, the RET+3:C allele is very highly conserved in all 9 mammalian species examined (FIG. 5) and it is the derived polymorphic allele (RET+3:T) that is overtransmitted. We postulate that RET+3 is the most likely site of the disease variation.

It was queried whether HSCR-susceptibility within this locus can be explained by RET alone or whether additional common variants might be present at GALNACT-2 or RASGEF1A. The Exhaustive Allelic TDT (EATDT), a novel method to iteratively and successively test all possible haplotypes of all possible sizes for association with HSCR^{12,13} was used. Seventeen haplotypes are significantly associated with HSCR but they have two critical properties (FIG. 1b): (1) no associated haplotype is limited to markers across GALNACT-2 or RASGEF1A; (2) all haplotypes involve RET SNPs alone, particularly those in intron 1. These results strongly suggest a role for a single, common variant within RET. Since all but one haplotype involves RET+3, it was concluded that the HSCR association arises from RET+3 (1) being in tight LD with a yet unknown disease-susceptibility variant, (2) being the disease-causing mutation alone, or (3) being a disease-causing variant that acts synergistically with additional disease variants on the associated haplotype.

### Comparative Genomics to Define Functional Elements

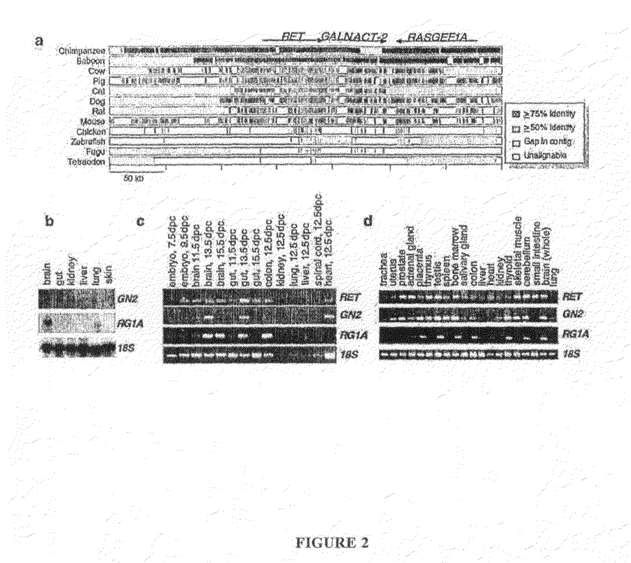

The finding of association across an intron suggested the need to identify functional elements within the RET locus. Systematic comparisons of orthologous sequences can uncover coding and non-coding functional elements on the assumption that such regions evolve slower than non-functional (neutral) sequences.^{14, 11, 10, 9}. The genomic sequence of a ˜350-kb segment encompassing human RET was obtained and compared with the orthologous intervals in 12 non-human vertebrates. Multi-species conserved sequences (MCSs) were identified as the intersection of elements which satisfied the criteria of Bray¹⁵ and Margulies¹⁶. Synteny is preserved across this interval in all vertebrates examined, although the fraction of sequence that can be aligned with the human sequence decreases with increasing evolutionary distance (FIG. 2a).

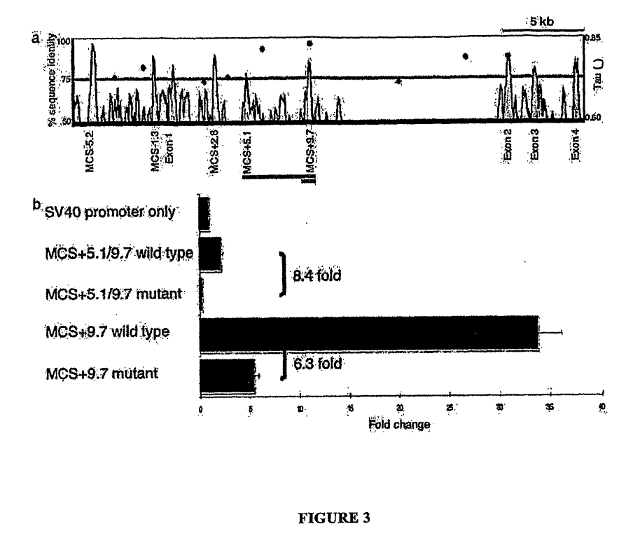

A total of 84 MCSs were identified (Table 3), with 44% (37/84) of the identified MCSs corresponding to exons of RET, GALNACT-2 and RASGEF1A. The remaining 47 MCSs are likely non-coding since no matching cDNA sequence or open reading frame greater than 20 amino acids in length was found. We identified 5 such elements within the most highly associated 27.6-kb around RET intron 1 (MCS−5.2, MCS−1.3, MCS+2.8, MCS+5.1 and MCS+9.7, identified by their kb distance from the RET start site as (FIG. 3a)).

TABLE 3
Positions of all Identified MCSs^{a}

| Start | End | Length | Description | Exon # |
|---|---|---|---|---|
| 42750079 | 42750298 | 219 | Extragenic | |
| 42759068 | 42759363 | 295 | Extragenic | |
| 42765824 | 42766058 | 234 | Extragenic | |
| 42767294 | 42767649 | 355 | Extragenic | |
| 42847632 | 42847887 | 255 | Extragenic | |
| 42848019 | 42848428 | 409 | Extragenic | |
| 42849042 | 42849161 | 119 | Extragenic | |
| 42851086 | 42851421 | 335 | Extragenic | |
| 42855277 | 42855460 | 183 | Extragenic | |
| 42856618 | 42856867 | 249 | RET coding | 1 |
| 42859464 | 42859741 | 277 | RET intron | |
| 42861719 | 42861898 | 179 | RET intron | |
| 42866040 | 42866290 | 250 | RET intron | |
| 42879799 | 42880213 | 414 | RET coding | 2 |
| 42881785 | 42882105 | 320 | RET coding | 3 |
| 42884371 | 42884649 | 278 | RET coding | 4 |
| 42885781 | 42885995 | 214 | RET coding | 5 |
| 42888446 | 42888719 | 273 | RET coding | 6 |
| 42890659 | 42890942 | 283 | RET coding | 7 |
| 42891570 | 42891681 | 111 | RET coding | 8 |
| 42892246 | 42892423 | 177 | RET coding | 9 |
| 42893017 | 42893163 | 146 | RET coding | 10 |
| 42893936 | 42894201 | 265 | RET coding | 11 |
| 42896029 | 42896206 | 177 | RET coding | 12 |
| 42897767 | 42897941 | 174 | RET coding | 13 |
| 42898979 | 42899202 | 223 | RET coding | 14 |
| 42899531 | 42899649 | 118 | RET coding | 15 |
| 42901360 | 42901477 | 117 | RET coding | 16 |
| 42903071 | 42903295 | 224 | RET coding | 17 |
| 42904333 | 42904442 | 109 | RET coding | 18 |
| 42904882 | 42905016 | 134 | RET intron | |
| 42906007 | 42906455 | 448 | RET coding | 19 |
| 42906737 | 42907024 | 287 | RET intron | |
| 42907345 | 42907818 | 473 | RET coding | 20 |
| 42908007 | 42908108 | 101 | RET intron | |
| 42908320 | 42908431 | 111 | RET intron | |
| 42908795 | 42908892 | 97 | RET intron | |
| 42908915 | 42909011 | 96 | RET coding | |
| 42909047 | 42909213 | 166 | RET 3′ UTR | 21 |
| 42909233 | 42909531 | 298 | RET 3′ UTR | 21 |
| 42909623 | 42909898 | 275 | RET 3′ UTR | 21 |
| 42920171 | 42920270 | 99 | GALNACT-2 intron | |
| 42932033 | 42932131 | 98 | GALNACT-2 intron | |
| 42933380 | 42933507 | 127 | GALNACT-2 intron | |
| 42934314 | 42935286 | 972 | GALNACT-2 coding | 2 |
| 42935414 | 42935519 | 105 | GALNACT-2 intron | |
| 42938093 | 42938420 | 327 | GALNACT-2 coding | 3 |
| 42938436 | 42938675 | 239 | GALNACT-2 intron | |
| 42939392 | 42939528 | 136 | GALNACT-2 intron | |
| 42939590 | 42939779 | 189 | GALNACT-2 intron | |
| 42939849 | 42940073 | 224 | GALNACT-2 coding | 4 |
| 42941406 | 42941679 | 273 | GALNACT-2 intron | |
| 42941954 | 42942112 | 158 | GALNACT-2 intron | |
| 42942628 | 42942804 | 176 | GALNACT-2 intron | |
| 42943088 | 42943227 | 139 | GALNACT-2 intron | |
| 42943232 | 42943566 | 334 | GALNACT-2 coding | 5 |
| 42943578 | 42943752 | 174 | GALNACT-2 intron | |
| 42946387 | 42946624 | 237 | GALNACT-2 coding | 6 |
| 42962682 | 42963286 | 604 | GALNACT-2 coding | 8 |
| 42963538 | 42963668 | 130 | GALNACT-2 3′ UTR | 8 |
| 42964122 | 42964240 | 118 | GALNACT-2 3′ UTR | 8 |
| 42964498 | 42964819 | 321 | GALNACT-2 3′ UTR | 8 |
| 42974002 | 42974194 | 192 | RASGEF1A 3′ UTR | 11 |
| 42974198 | 42974366 | 168 | RASGEF1A 3′ UTR | 11 |
| 42974969 | 42975687 | 718 | RASGEF1A 3′ UTR | 11 |
| 42975877 | 42976015 | 138 | RASGEF1A coding | 10 |
| 42976428 | 42976562 | 134 | RASGEF1A coding | 9 |
| 42977449 | 42977664 | 215 | RASGEF1A coding | 8 |
| 42978384 | 42978654 | 270 | RASGEF1A coding | 7 |
| 42979069 | 42979280 | 211 | RASGEF1A coding | 6 |
| 42979602 | 42979699 | 97 | RASGEF1A coding | 5 |
| 42980068 | 42980400 | 332 | RASGEF1A coding | 4 |
| 42981223 | 42981454 | 231 | RASGEF1A coding | 3 |
| 42982718 | 42982868 | 150 | RASGEF1A coding | 2 |
| 42985367 | 42985589 | 222 | RASGEF1A coding | 1b |
| 42994619 | 42994928 | 309 | RASGEF1A intron | |
| 42998282 | 42998387 | 105 | RASGEF1A intron | |
| 42998429 | 42998591 | 162 | RASGEF1A intron | |
| 42999227 | 42999321 | 94 | RASGEF1A intron | |
| 43041678 | 43041839 | 161 | RASGEF1A intron | |
| 43043917 | 43044130 | 213 | RASGEF1A intron | |
| 43044601 | 43044684 | 83 | RASGEF1A intron | |
| 43045824 | 43046021 | 197 | RASGEF1A intron | |
| 43046209 | 43046493 | 284 | RASGEF1A 5′ UTR | 1a |

^{a}Positions on human chromosome 10 are given relative to build 34 (July 2003) of the genome; see www.genome.ucsc.edu

Although GALNACT-2 and RASGEF1A are unlikely to harbor common HSCR variants they might carry rare mutations and be important in HSCR, just as some of the 126 patients we studied also have rare RET mutations. To test their involvement in enteric development and HSCR, their temporal and spatial expression in humans and mice was characterized. Transcription of RASGEF1A is limited to brain and several tissues (bone marrow, testis, colon, and placenta) with high replicative capacity (FIG. 2 b, c, d). RET and GALNACT-2 share overlapping, nearly ubiquitous postnatal expression patterns. Importantly, GALNACT-2 and RASGEF1A are both highly expressed at 13.5 dpc, coincident with peak RET expression and colonization of the gut by neural crest-derived neuronal precursors (FIG. 2c), a feature disrupted in HSCR. Consequently, GALNACT-2 and RASGEF1A expression patterns are consistent with a potential role in enteric neural crest migration. The analysis of morpholino-based gene knockdowns of the orthologous genes in zebrafish has, however, uncovered only mid-gastrulation defects in convergence and extension for Galnact-2 and central nervous system neuronal cell death by 24 hours post fertilization for Rasgef1a (data not shown). In contrast, similar disruption of RET results in incomplete colonization of the digestive tube by enteric neurons^{17,18}. These functional analyses cannot exclude either GALNACT-2 or RASGEF1A as HSCR candidate genes as the observed embryonic lethality occurred prior to the onset of neural crest cell migration into the digestive tube. However, genetic association tests have excluded the occurrence of a common mutation at GALNACT-2 or RASGEF1A contributing to HSCR.

### MCS+9.7 Functions as an Enhancer In Vitro

Although MCS+9.7 is likely a functional element, the specific function of this sequence and the mechanism by which it exhibits a deleterious effect is not known. MCS+9.7 demonstrates a minimum identity of 72.5% with all mammalian species examined. No predicted structural/regulatory RNAs were identified in MCS+9.7 using the QRNA algorithm¹⁹. The MCS+9.7 sequence includes a gamut of predicted transcription factor binding sites (Table 4), including two retinoic acid response elements (RARE) within four nucleotides on either side of the RET+3 site. However, no predicted binding sites are disrupted directly by the mutant RET+3:T allele or the alleles at the rs2506004 and rs2506005 sites. Importantly, retinoic acid has already been documented as a negative and a positive regulator of RET expression in cardiac and renal development, respectively^{20,21}. Furthermore, exogenous retinoic acid delays hindgut colonization by RET-positive enteric neuroblasts and results in ectopic RET expression during embryogenesis²². Although the mutation(s) does not introduce or destroy a predicted RARE, it may introduce a novel site that permits competition with, or reduces access to, the neighboring predicted RAREs. Clearly, the ultimate proof of disease-causation will require the synthesis of the trait, from one or all three of the MCS+9.7 variants, in an appropriate model organism.

TABLE 4
Predicted transcription factor binding sites in
MCS + 9.7^{a}

| | Start | | |
|---|---|---|---|
| Factor | nucleotide^{b} | Length | Sequence |
| Sp1 | 42866044 | 6 | GGGGCC |
| RAR | 42866048 | 10 | CCAGTGACCC |
| RORalpha1 | 42866051 | 13 | GTGACCCTTACAT |
| NP-III | 42866051 | 6 | GTGACC |
| AP-1 | 42866052 | 6 | TGACCC |
| RAR-alpha1 | 42866052 | 6 | TGACCC |
| SRF_Q6 | 42866054 | 14 | ACCCTTACATGGTC |
| SAP-1 | 42866056 | 10 | CCTTACATGG |
| SRF | 42866056 | 10 | CCTTACATGG |
| myc-CF1 | 42866060 | 6 | ACATGG |
| RC2 | 42866064 | 7 | GGTCATC |
| RAR-alpha1 | 42866064 | 16 | GGTCANNNNNNGGtCA |
| CACCC-binding | | | |
| factor | 42866083 | 6 | GGGTGG |
| Sp1 | 42866083 | 6 | GGGTGG |
| CP2 | 42866088 | 7 | GCCAGTC |
| LVa | 42866095 | 6 | CTGTTC |
| NF-1 | 42866101 | 6 | AGCCAG |
| NF-1 | 42866109 | 6 | CTTGCC |
| NF-1 | 42866117 | 7 | AGGAAAG |
| SBF-1 | 42866123 | 14 | GAAATTAATTATAA |
| N-Oct-3 | 42866125 | 7 | MATWAAT |
| MEF-2 | 42866127 | 10 | TTAATTATAA |
| TBP | 42866127 | 7 | TTAATTA |
| RSRFC4 | 42866128 | 8 | TAWWWWTA |
| IF2 | 42866136 | 10 | ACCTAATTGG |
| CCAAT-binding | | | |
| factor | 42866141 | 6 | ATTGGC |
| NF-1/L | 42866142 | 6 | TTGGCA |
| c-Ets-1_54 | 42866146 | 13 | CAGTTTCCTTTGC |
| NFAT_Q6 | 42866146 | 12 | CAGTTTCCTTTG |
| IBP-1 | 42866146 | 11 | CAGTTTCCTTT |
| PEA3 | 42866149 | 6 | TTTCCT |
| c-Ets-2 | 42866150 | 6 | TTCCTT |
| Oct-1 | 42866150 | 13 | TTCCTTTGCATAG |
| Pit-1a | 42866155 | 7 | TTGCATA |
| EFII | 42866156 | 6 | TGCATA |
| Elk-1 | 42866162 | 16 | GAAGCCGGAAGCAACT |
| c-Myb | 42866173 | 6 | CAACTG |
| Sp1 | 42866184 | 9 | KRGGCKRRK |
| GATA-1 | 42866192 | 6 | TGATTA |
| AP-1 | 42866192 | 7 | TGATTAA |
| Zen-2 | 42866193 | 12 | GATTAACTCTGC |
| Eve | 42866193 | 12 | GATTAACTCTGC |
| HNF-1 | 42866194 | 6 | ATTAAC |
| ITF-2 | 42866203 | 10 | GCAGCAGCTG |
| Myf-5 | 42866204 | 9 | CAGCAGCTG |
| MyoD | 42866204 | 11 | CAGCAGCTGGG |
| AP-4 | 42866204 | 9 | CAGCAGCTG |
| E2A | 42866206 | 7 | GCAGCTG |
| Myogenin | 42866206 | 7 | GCAGCTG |
| RFX2 | 42866207 | 6 | CAGCTG |
| Tal-1 | 42866207 | 6 | CAGCTG |
| AP-4 | 42866207 | 6 | CAGCTG |
| XPF-1 | 42866207 | 6 | CAGCTG |
| C/EBPbeta | 42866210 | 7 | CTGGRAA |
| Ik-1 | 42866211 | 6 | TGGGAA |
| EFII | 42866217 | 6 | ATTGCA |
| c-Myb | 42866221 | 6 | CAGTTG |
| C/EBPalpha | 42866223 | 6 | GTTGGG |
| Ttk_88K | 42866226 | 10 | GGGCAGGAGC |
| Sp1 | 42866226 | 6 | GGGCAG |
| Myogenin | 42866228 | 7 | GCAGGAG |
| PEA3 | 42866242 | 6 | CATCCT |
| Adf-1 | 42866251 | 16 | CAGGCCGCTGCAGCTG |
| ITF-2 | 42866257 | 10 | GCTGCAGCTG |

^{a}Based on TRANSFAC 4.0 predictions (http://www.cbil.upenn.edu/cgi.bin/tess) of p ≦ 10⁻² and La ≧ 10.
^{b}Specified positions are in reference to chromosome 10, build 34 (July 2003) of the human genome.

Based on its location, we predicted that the MCS+9.7 element functions as a transcriptional enhancer or suppressor. Using transient transfection assays, we tested the function of two RET intron 1 constructs in the mouse neuroblastoma cell line Neuro-2a. Amplicons containing MCS+9.7 and MCS+5.1/+9.7 show enhancer activity in this cell line (FIG. 3b), although this activity in HeLa cells is negligible (data not shown), suggesting that the activity of MCS+9.7 is cell-type dependent. Importantly, amplicons harbouring the mutant allele demonstrate significantly lower enhancer activity (6- to 8-fold decrease) than those containing the wild type allele (t-test, p value ≦0.001). These data suggest that the mutation lies within and compromises the activity of an enhancer-like sequence in RET intron 1. RET coding sequence mutations in HSCR are always loss-of-function alleles. Thus our finding that the RET+3 mutation decreases transcription is consistent with HSCR biology. We can localize the enhancer function, and the genetic change which diminishes that function, to the 900-nt fragment tested in the MCS+9.7 construct. Within this region exist three segregating sites (rs2506005, RET+3 and rs2506004) in complete LD. In principle, any one of these three sites, or their combination, can be the disease susceptibility factor.

### World-Wide Distribution of MCS+9.7 Variants

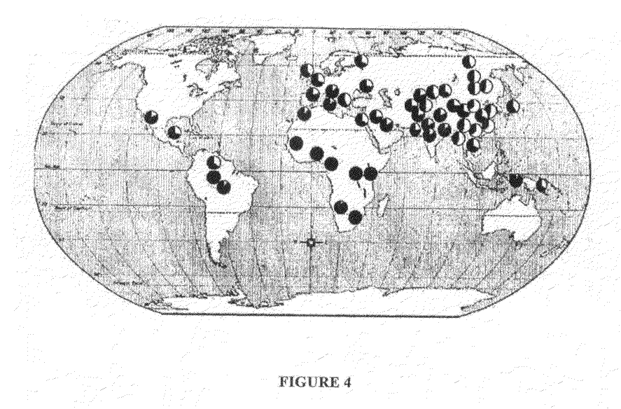

The global distribution of the RET+3:T allele was determined by genotyping individuals from 51 unselected populations. The mutant T allele is virtually absent within Africa (<0.01), has intermediate frequency in Europe (0.25) but reaches high frequency (0.45) in Asia (FIG. 4). Additionally, we generated haplotypes for 7 SNPs from 60 individuals, each from Africa, Europe and Asia, derived from the above world-wide set and compared them to haplotypes from HSCR patients (Table 5). Haplotypes bearing the RET+3:T allele likely have a single origin, sometime after modern humans emerged from Africa. Intriguingly, the high frequency of the RET+3:T allele, and the susceptibility haplotype, in East Asia correlates with an increased incidence of short segment HSCR among Asian newborns (3.1 vs. 1.5 per 10,000 births in Asian American versus European American births in California between 1983 and 1997; C. Torfs, 1998; personal communication). This same haplotype has a 66% frequency among Chinese sporadic HSCR patients 5; consequently, a 2-fold increase in the mutant allele frequency translates into a roughly 2-fold increase in disease incidence. We suspect that RET+3:T is a marker for short segment HSCR since the low frequency of the RET+3:T allele in Africa correlates with a lower frequency of short segment HSCR among African Americans².

Haplotype frequencies in Africa, Asia, Europe and HSCR cases.

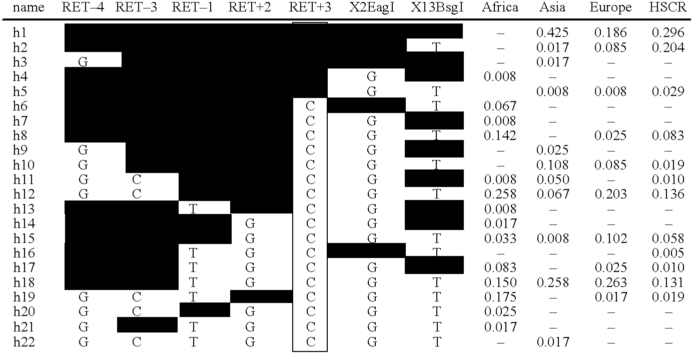

60 individuals were selected from the HGDP samples representing each continent.
HSCR: all available HSCR cases.
Haplotypes were reconstructed using PHASE⁴¹. For each SNP, the HSCR-associated allele is highlighted in yellow. Position of RET + 3 is indicated by the red box.
—indicates the haplotype was not observed among the chromosomes genotyped.

These data strongly argue that among the three SNPs within MCS+9.7 only the RET+3 variant is the susceptibility mutation. The associated alleles at rs2506005, RET+3 and rs2506004 are the ancestral, derived and ancestral alleles, respectively. Given our knowledge of human evolution and that the susceptibility haplotype has 1% frequency in Africa, the ancestral haplotype (with ancestral alleles at each SNP) was virtually extinct within Africa until it rose in frequency with the occurrence of the RET+3:T mutation.

This finding of a common allele that rapidly increased in frequency but is associated with a disease predisposition can be explained in one of three ways: (1) recurrent mutations from the wild type to the same deleterious mutant; (2) chance increase by genetic drift; and (3) selective advantage of the mutation in heterozygotes. The finding of a common haplotype suggests that the first explanation is unlikely. To distinguish between the two remaining alternatives, we performed two analyses: (a) we estimated an FST value of 0.027; (b) we compared our world-wide mutant allele distribution (summarized as allele frequency <5% in Africa, >25% in Europe, and >40% in China/Japan) to that of 8,247 SNPs from the ENCODE loci²³. Only 38 sites (0.46%) show the observed or a more extreme pattern, strongly suggesting selective advantage to the mutation.

If polymorphisms make substantial contributions to common disorders then a significant fraction of them must have been exposed to selection. It is not surprising, then, that a majority of common disease associations involve alleles that provided (α-globin, β-globin²⁴, G6PD²⁵⁻²⁷, HLA²⁸, Fy²⁹ and other variants in malaria), or are suspected of providing (CCRΔ32 in HIV infection³⁰⁻³²), a survival advantage to humans. Thus, many common variants in currently common disorders perhaps stem from alleles that were, or are, protective for another phenotype, providing mechanistic support to the common variant, common disease model of genetic disease^{33,34}.

Prior to the advent of corrective surgical methodologies in the 1950s, HSCR was a uniformly fatal disorder, necessitating positively acting selective forces to maintain this deleterious allele at high frequency. Our demonstration that the RET+3:T allele is a derived allele that is virtually absent in Africa but rose to a frequency of 0.25 in Europe and 0.45 in Asia in 100,000 years or less is indicative of such a selective force. RET is a tyrosine kinase receptor on the surface of neuroblasts, and many other cell types, and it is not inconceivable that it might be a target of pathogen entry, such as the chemokine receptors involved in HIV and malaria.

### Genetic Properties of the RET+3 Susceptibility Allele

A pervasive feature of HSCR is the marked gender difference in expression and incidence, with males being four times more likely to be affected than females. These sex differences could arise from mutations on the X chromosome, but genome-wide mapping studies^{1,7} have consistently failed to identify an X-linked gene. Consequently, we tested whether the RET+3 variant at MCS+9.7 shows sex-specific effects. As shown in Table 1, transmission frequency of the associated allele in the RET region is always smaller to affected daughters than to affected sons, with rare exceptions at non-significant SNPs. Indeed, given the lower female penetrance, there were fewer affected daughters than sons in our sample, and among them only the mutant SNP (boys: r=0.86, p=3.7×10⁻¹¹; girls r=0.68, p=0.02) and the SNP at 1.1SfcI, 3.3 kb away, are statistically significantly different from 0.50. Nevertheless, a trend test for a difference in male and female offspring transmission frequency is highly significant and estimates the male-to-female transmission ratio to be ˜2 (p=0.0007). Thus, the genetic effect at MCS+9.7 is significantly greater in sons than in daughters.

Two other features of the RET+3 mutation display sex differences consistent with the greater incidence in males than females. First, as shown in Table 2, the transmission frequency to affected sons and daughters leads to a 5.7-fold and 2.1-fold increase in susceptibility in males and in females, respectively, assuming a multiplicative model for penetrance. Second, genotype frequencies of affected individuals can be used to estimate the penetrance, which varies between 6.2×10⁻⁵ and 1.8×10⁻³ (Table 2) and is considerably smaller than that for long segment HSCR. Our finding of gender differences in penetrance is consistent with the greater incidence of HSCR in males. For all traits demonstrating gender-specific differences in incidence, affected individuals from the less frequently affected sex (females for HSCR) have a higher mean susceptibility. Therefore, when we consider the totality of all susceptibility loci, we expect females with HSCR to carry more susceptibility alleles than their male counterparts³⁵. It follows that the penetrance of any specific mutation must be lower for the lesser affected sex, as observed here.

To assess the genetic impact of this common mutation we estimated the proportion of the total variance in susceptibility that the RET+3 mutation explains. Surprisingly, only 2.63% and 1.14% of the variation is explained by the action of this mutation in males and females, respectively (Table 2). This is in contrast to the meagre 0.1% of the total variance in susceptibility explained by all known coding mutations at RET². Consequently, the MCS+9.7 enhancer mutation explains a 10 to 20-fold greater susceptibility variation than all other known RET mutations. However, our findings also caution that a considerable number of additional loci may remain to be identified.

TABLE 2
Genetic characteristics of the RET enhancer mutation

| | Observed genotype | | | Penetrance | |
|---|---|---|---|---|---|
| | counts† | | Expected | (×10⁵)§ | |
| Genotype | Males | Females | frequency‡ | Males | Females |
| CC | 40 | 15 | 0.58 | 16.1 ± 2.2 | 6.2 ± 0.9 |
| CT | 50 | 17 | 0.97 | 34.5 ± 3.8 | 6.4 ± 1.3 |
| TT | 37 | 26 | 0.08 | 175.0 ± 22.9 | 35.9 ± 8.0 |
| Risk ratio (γ)# | | | | 5.7 | 2.1 |
| Variation (%) | | | | 2.63 | 1.14 |

A final interesting gender difference is that the mutant allele arises from mothers and fathers in 35 and 18 of the 53 informative families, respectively. This is significantly different from expectation (p=0.02) and similar to the effect we previously observed in linkage analysis of RET in a different series of families⁷. The cause of this bias is unknown since RET is not known to be imprinted; however, whether RET shows specific imprinting in neuroblasts is unknown.

The identification of the RET+3 mutation was aided by comparative sequence analysis and emphasized by its likely selection. This finding has several implications for genetic analyses of both Mendelian and complex disease. Mutation searches as described herein in human disease include both coding sequences of genes and neighboring non-coding elements. For example, non-coding mutations may conspire with mutations at additional genes for disease to occur, but also in rare Mendelian phenotypes where 10-15% of patients can have no recognized mutations despite incontrovertible evidence for a single known gene. Not all mutations for rare diseases are required to be rare or have 100% penetrance. Thus, the criterion of identifying mutations as sequence changes that are absent in controls may not be appropriate for a significant fraction of alterations and may exclude legitimate mutations. The inheritance patterns of single gene traits due to common variants are somewhat different from those we have come to expect from rare Mendelizing mutations particularly when penetrance is not complete. Thus, apparent genetic heterogeneity in linkage or bilineal inheritance does not imply that mutations do not exist at a single locus.

A variety of non-coding elements are involved in transcription, translation, recombination, replication and repair, but full nature and function of these sequences is unknown. Comparative genomics provides an avenue for recognizing such elements in a generic way but this depends on the assumption that functional sites evolve recognizably slower than non-functional sites. These analyses have shown that only 1.5% of the human genome is devoted to coding exons and, as much as, 3% to conserved non-coding sequences³⁶, implying that the latter may be particularly important as sites of mutation. Provided herein is a molecular view to a multifactorial disorder: the most common mutation is non-coding, it has low (marginal) penetrance, the mutation has sex-dependent effects and explains only a small fraction of the total susceptibility to HSCR. Nevertheless, examples provided herein have three features that are relevant to the analysis of common complex disorders. First, although the known protein coding HSCR mutations have higher (51-72%) penetrance, their rarity in the population implies they explain only a minute fraction (0.1%) of the disorder. Thus, additional genes or environmental factors may explain disease incidence. Second, about 11% of our HSCR patients have known RET coding mutations in addition to carrying the RET+3:T variant. It is not unlikely that coding and non-coding mutation may act synergistically to affect disease penetrance, in other words, there may be more than one mutation per gene. Third, an enhancer mutation allows us to speculate that additional factors (proteins) interact with this element and can mitigate or attenuate its genetic effect on RET transcription. In sum, for common mutations, we expect that mutation penetrance will depend on other alleles and genes (genetic background), epigenetic effects (such as those associated with sex-linked gene dosage), or even the environment.

Patient samples. We genotyped trios with 126 probands, all their parents (of which 3 were affected) plus 24 unaffected siblings; for the penetrance studies we also genotyped additional probands for a total of 450 samples. All forms of HSCR (short segment, long segment, and total colonic aganglionosis) were represented in the patient sample. 11% of ascertained cases presented with additional anomalies, including defined neurocristopathies, chromosomal abnormalities (e.g., trisomy 21), and other defects. Ascertainment was conducted under informed consent approved by the Institutional Review Board of Johns Hopkins University School of Medicine. In addition to the HSCR patients and their families, we also genotyped 1,064 samples representing individuals from six continents from the CEPH Human Genome Diversity Panel (http://www.cephb.fr/HGDP-CEPH-Panel/;³⁷).

SNP genotyping. We selected SNPs with a minimum minor allele frequency of 10%, with physical map locations covering the three genes RET, GALNACT-2, RASGEF1A and emphasizing the associated region within RET⁸. From dbSNP, we selected SNPs with known heterozygosity and/or SNPs with both alleles observed twice (“double hit” SNPs); we used markers for which robust genotyping assays could be developed. All SNPs are referred to by their rs numbers. Genotypes were generated using the fluorogenic 5′ nuclease assay (Taqman, Applied Biosystems, Foster City, Calif.). A TECAN Genesis workstation was used for all liquid handling, thermal cycling was completed on MJ Research Tetrads, and end-point measurements were made on an ABI 7900. Genotypes were determined using SDS 2.1 (Applied Biosystems, Foster City, Calif.) and verified by the instrument operator. 10% of the samples (n=45) were genotyped in duplicate for all 30 markers; no discrepancies were observed among the 1,350 paired replicate genotypes.

Transmission disequilibrium test. The TDT chi square test statistic was used to identify significant deviation from the expected 1:1 Mendelian transmission¹¹. The transmission frequency (τ) from heterozygous parents to offspring was estimated from all family genotype data at each SNP by maximum likelihood. We assumed either a (i) single τ, (ii) τ different by parent gender (τₘ, τ_{f}), or (iii) different transmission rates to male (b) and female (g) children (τ_{b}, τ_{g}). Chi square tests with 1 degree of freedom based on the appropriate likelihood ratio were used to test whether τ=½, τₘ=τ_{f} or τ_{b}=τ_{g}.

Haplotype reconstruction and Exhaustive Allelic TDT (EATDT). For family based samples, haplotypes were inferred using hap2, a method that combines traditional family-based reconstructions with population-based linkage disequilibrium information to achieve extremely accurate reconstruction within nuclear families¹². Haplotypes for control HGDP individuals were reconstructed with PHASE³⁸, Exhaustive allelic transmission disequilibrium tests (EATDT) were performed, following haplotype reconstruction, for all sliding windows of all numbers of SNPs at all positions. Within each window of any size, all observed haplotypes were tested for association by the TDT. To assess overall significance, while accounting for multiple tests, 10⁸ permutations were performed to estimate a p value.

Re-sequencing. Three re-sequencing experiments were performed and analyzed to identify novel SNPs: (1) DNA chip-based re-sequencing³⁹ of the non-repeat sequence in a 90-kb interval containing RET in 32 Mennonites (15 HSCR cases and 17 controls); (2) re-sequencing MCSs within RET intron 1 in 22 HSCR patients from families with RET-linkage but no identified coding sequence mutations; (3) re-sequencing 9 kb around RET+3 in 4 and 8 individuals each homozygous for the RET+3:T and the RET+3:C allele, respectively. These analyses identified numerous rare and novel SNPs, additional low frequency SNPs existing in dbSNP, and a high frequency SNP within intron 1 enriched in patients, RET+3. In addition to RET+3, we identified variants within three additional intron 1 conserved elements (see later) by re-sequencing in HSCR patients.

Allele distribution at ENCODE loci. The ENCODE project²³ has identified all segregating sites at 5 loci on human chromosomes 2p16.3, 2q37.1, 4q26, 7q21.13 and 7q31.33 each ˜500 kb in length. All SNPs were genotyped in the HapMap samples from four populations, namely, Utah CEPH, Yoruba from Ibadan, Nigeria, Han Chinese from Beijing and Japanese from Tokyo, Japan (www.HapMap.org). We estimated allele frequencies at 8,247 SNPs in the three continental regions (Europe, Africa and Asia; 60 independent individuals each) and compared them to the RET+3:T allele. We estimated the probability of observing allele frequency <5% in Yoruba, >25% in Europe, and >40% in China/Japan in all 8,247 SNPs as 0.0046. To reduce effects of LD, we sampled every second (4,121 SNPs), fourth (2,059 SNPs), eighth (1,028 SNPs) and sixteenth (512 SNPs) SNP to obtain probabilities of 0.0036, 0.0049, 0.0068 and 0.0059, respectively. An identical analysis using the FST statistics gave a p-value of 0.027 (0.023-0.029).

Estimating the susceptibility variance due to a polymorphism. We assume that the variation in susceptibility to HSCR is multifactorial and parametrized as described in¹³. The three genotypes at the susceptibility locus are AA, Aa and aa with frequencies p², 2pq, q², respectively; means of 0, dt and t, respectively (t=displacement; d=degree of dominance); residual variance of 1 arising from additional genes and the environment. Genotype-specific susceptibility distributions are Gaussian, and all measurements on the susceptibility scale are in standard deviation units. Affection arises whenever the susceptibility exceeds a biological threshold Z so that genotype-specific penetrance is the integrated Gaussian density above Z.

Penetrance of the CC, CT and TT genotypes at RET+3 (C=wild type; T=mutant) can be estimated using inverse probability given the observed numbers of affecteds with these genotypes, assuming a disease incidence (S-HSCR and L-HSCR are 80% and 20% of the total incidence of 1/5,000) and the mutant allele frequency (q=0.24 from the untransmitted chromosomes in 252 parents of probands). Consequently, we can estimate Z from the CC penetrance, and given the threshold we can estimate the susceptibility means from the two other genotype distributions; estimation was by the maximum likelihood method. Finally, the variance in susceptibility between genotypes can be calculated from the three estimated means. Multi-species genomic sequences. Genomic sequences orthologous to a 350-kb region encompassing the RET gene were generated from multiple species. Publicly available genomic sequences data were used for human and mouse (Hg16, chr10: 42700000-43050000 (human) and Mm3, chr6: 118646816-119036816. Bacterial artificial chromosome (BAC) clones from seven non-human vertebrates (chimpanzee, baboon, cow, pig, cat, dog, and rat) were isolated by screening BAC libraries with ‘universal’ hybridization probes⁴³. For non-mammalian organisms (chicken, zebrafish, fugu, and tetraodon), species-specific probes were designed from available gene sequence. Following mapping, selected BACs were sequenced by the NISC Comparative Sequencing Program. Additionally, orthologous chicken sequences were obtained from the whole-genome assembly available at http://genome.ucsc.edu.

Comparative sequence analysis. Sequences were aligned and visualized with mVISTA^{18,44} and MultiPipMaker⁴⁵. Multi-species conserved sequences (MCSs) were identified with the algorithm of Margulies et al. (2003). Briefly, this method utilizes multiple alignments (MultiPipMaker) and calculates conservation scores for 25-nt overlapping windows with 1-nt increments. We used 5% of the reference sequence as the appropriate cut-off for conserved sequence identification¹⁹ as 5% of the human genome is presumed to be under natural selection³⁹. We considered the overlapping set of mVISTA:MCS elements because MCSs alone can fragment known functional units (e.g. exons) into multiple smaller fragments. For mVISTA analysis, we chose a pairwise comparison between mouse and human. Importantly all elements identified between comparison with human and any other vertebrate were represented by the mouse-human comparison suggesting this pairwise comparison is fully representative of the conserved elements in the region. MCSs included >98.9% of all nucleotides within these exons and less than 0.59% of ancient repeat sequence in the region. The summed lengths of all identified MCSs was 19.8-kb.

MCSs identified all exons encoding RET, GALNACT-2 and RASGEF1A. No additional genes were identified 5′ to RET in the region we obtained and sequenced. The human genome sequence (http://genome.ucsc.edu: build 35) predicts that the gene most proximal to the 5′ end of RET, BMS1L, a putative ribosome biogenesis protein, lies 246-kb upstream of RET exon 1.

Expression analysis. Temporal and spatial expression patterns of RET, GALNACT-2, and RASGEF1A were established by reverse transcriptase-polymerase chain reaction (RT-PCR) and northern blotting. Human total RNA samples were from the Clontech™ (Palo Alto, Calif.) MTC human RNA panels. Embryonic and post-natal mouse RNAs were isolated from timed matings between 129SvImJ mice. All animal studies were conducted under protocols approved by the Johns Hopkins University Animal Care and Use Committee. All primer and probe sequences used in this study are available at http://chakravarti.igmjhmi.edu/pro_site/projects/RET_Nature2005.

Luciferase assays. DNA samples from individuals homozygous for the T and C alleles at RET+3 were amplified, sequenced to verify their composition, and cloned into the Gateway pDONR™221 entry vector per the manufacturer's protocol. Amplicons were subcloned into a Sma I site in a Gateway® modified pGL3 (Promega™, Madison, Wis.) firefly luciferase vector containing an SV40 promoter and complete firefly luciferase open reading frame. Plasmids containing only the SV40 promoter and luciferase reporter (pDSma_promoter) and plasmids without the SV40 promoter (pDSma_control) served as experimental control vectors.

The neuroblastoma cell line (Neuro-2a, ATCC# CCL-131) was cultured according to ATCC protocols. Neuro-2a derive from a peripheral neuronal population that expresses the products of several HSCR genes (Ret, Ednrb, and Sox10), the neural crest-specific p75^{NTR} gene, and the neuronal marker Dbh (data not shown). Approximately 1×10⁶ Neuro-2a cells were co-transfected (Lipofectamine Plus™, Invitrogen, Carlsbad, Calif.) with 0.4 μg of the appropriate pDSma firefly luciferase plasmid and 0.01 μg phRL-SV40 control Renilla luciferase plasmid; Renilla luciferase control plasmid was used to normalize all data points. Dual Luciferase® Assays (Promega, Madison, Wis.) were performed 24 hours after transfection according to manufacturer's protocols (Monolight® 2010, Analytical Luminescence Laboratories, CA). Fold change was calculated relative to samples transfected with the promoter-only construct (pDSma_promoter). Statistical significance was determined using a 2-tailed t-test assuming unequal variances.

Accession numbers for genomic sequences reported in this paper: Hg16, chr10:42700000-43050000 (human); Mm3, chr6:118646816-119036816 (mouse), AC125509 and AC125512 (baboon), AC124166 (cat), AC138567 (chicken), RP43-171H18 (chimpanzee), AC124163 and AC124164 (cow), AC123973 (dog), AC124911 and AC125500 (fugu), AC122156 and AC124165 (pig), AC114881 (rat), AC135546 (tetra), and AC124155 (zebrafish).

### REFERENCES

1. Bolk, S. et al. A human model for multigenic inheritance: phenotypic expression in Hirschsprung disease requires both the RET gene and a new 9q31 locus. Proc Natl Acad Sci USA 97, 268-73 (2000).
2. Chakravarti, A. & Lyonnet, S. Hirschsprung disease (eds. Scriver, C. R. & al., e.) (McGraw-Hill, New York, 2001).
3. Carrasquillo, M. M. et al. Genome-wide association study and mouse model identify interaction between RET and EDNRB pathways in Hirschsprung disease. Nat Genet. 32, 237-44 (2002).
4. Borrego, S. et al. RET genotypes comprising specific haplotypes of polymorphic variants predispose to isolated Hirschsprung disease. J Med Genet 37, 572-8 (2000).
5. Garcia-Barcelo, M. M. et al. Chinese patients with sporadic Hirschsprung's disease are predominantly represented by a single RET haplotype. J Med Genet 40, e122 (2003).
6. Sancandi, M. et al. Single nucleotide polymorphic alleles in the 5′ region of the RET proto-oncogene define a risk haplotype in Hirschsprung's disease. J Med Genet 40, 714-8 (2003).
7. Gabriel, S. B. et al. Segregation at three loci explains familial and population risk in Hirschsprung disease. Nat Genet 31, 89-93 (2002).
8. McCallion, A. S. et al. Genomic Variation in Multigenic Traits: Hirschsprung Disease (ed. Stillman, B.) (CSHL Press, Cold Spring Harbor, 2003).
9. Uyama, T. et al. Molecular cloning and expression of a second chondroitin N-acetylgalactosaminyltransferase involved in the initiation and elongation of chondrbitin/dermatan sulfate. J Biol Chem 278, 3072-8 (2003).
10. Sato, T. et al. Molecular cloning and characterization of a novel human beta 1,4-N-acetylgalactosaminyltransferase, beta 4GalNAc-T3, responsible for the synthesis of N,N′-diacetyllactosediamine, galNAc beta 1-4GlcNAc. J Biol Chem 278, 47534-44 (2003).
11. Spielman, R. S., McGinnis, R. E. & Ewens, W. J. Transmission test for linkage disequilibrium: the insulin gene region and insulin-dependent diabetes mellitus (IDDM). Am J Hum Genet 52, 506-16 (1993).
12. Lin, S., Chakravarti, A. & Cutler, D. J. Haplotype and Missing Data Inference in Nuclear Families. Genome Res in press (2004).
13. Lin, S., Chakravarti, A. & Cutler, D. J. Exhaustive allelic transmission disequilibrium tests as a new approach to genome-wide association studies. Nat Genet 36, 1181-8 (2004).
14. Loots, G. G. et al. Identification of a coordinate regulator of interleukins 4, 13, and 5 by cross-species sequence comparisons. Science 288, 136-40 (2000).
15. Bray, N., Dubchak, I. & Pachter, L. AVID: A global alignment program. Genome Res 13, 97-102 (2003).
16. Margulies, E. H., Blanchette, M., Haussler, D. & Green, E. D. Identification and characterization of multi-species conserved sequences. Genome Res 13, 2507-18 (2003).
17. Shepherd, I. T., Pietsch, J., Elworthy, S., Kelsh, R. N. & Raible, D. W. Roles for GFRalpha1 receptors in zebrafish enteric nervous system development. Development 131, 241-9 (2004).
18. Shepherd, I. T., Beattie, C. E. & Raible, D. W. Functional analysis of zebrafish GDNF. Dev Biol 231, 420-35 (2001).
19. Rivas, E. & Eddy, S. R. Noncoding RNA gene detection using comparative sequence analysis. BMC Bioinformatics 2, 8 (2001).
20. Shoba, T., Dheen, S. T. & Tay, S. S. Retinoic acid influences the expression of the neuronal regulatory genes Mash-1 and c-ret in the developing rat heart. Neurosci Lett 318, 129-32 (2002).
21. Batourina, E. et al. Vitamin A controls epithelial/mesenchymal interactions through Ret expression. Nat Genet 27, 74-8 (2001).
22. Pitera, J. E., Smith, V. V., Woolf, A. S. & Milla, P. J. Embryonic gut anomalies in a mouse model of retinoic Acid-induced caudal regression syndrome: delayed gut looping, rudimentary cecum, and anorectal anomalies. Am J Pathol 159, 2321-9 (2001).
23. The ENCODE (ENCyclopedia Of DNA Elements) Project. Science 306, 636-40 (2004).
24. Haldane, J. B. S. The rate of mutation of human genes. Hereditas 35(Suppl), 267-273 (1948).
25. Allison, A. C. G-6-PD deficiency in red blood cells of East Africans. Nature 186, 531 (1960).
26. Allison, A. C. & Clyde, D. F. Malaria in African children with deficient erythrocyte glucose-6-phosphate dehydrogenase. Br Med J 5236, 1346-9 (1961).
27. Motulsky, A. Metabolic polymorphisms and the role of infectious disease in human evolution. Human Biology 32, 28 (1960).
28. Hill, A. V. et al. Common west African HLA antigens are associated with protection from severe malaria. Nature 352, 595-600 (1991).
29. Miller, L. H., Mason, S. J., Clyde, D. F. & McGinniss, M. H. The resistance factor to Plasmodium vivax in blacks. The Duffy-blood-group genotype, FyFy. N Engl J Med 295, 302-4 (1976).
30. Samson, M. et al. Resistance to HIV-1 infection in caucasian individuals bearing mutant alleles of the CCR-5 chemokine receptor gene. Nature 382, 722-5 (1996).
31. Dean, M. et al. Genetic restriction of HIV-1 infection and progression to AIDS by a deletion allele of the CKR5 structural gene. Hemophilia Growth and Development Study, Multicenter AIDS Cohort Study, Multicenter Hemophilia Cohort Study, San Francisco City Cohort, ALIVE Study. Science 273, 1856-62 (1996).
32. Huang, Y. et al. The role of a mutant CCR5 allele in HIV-1 transmission and disease progression. Nat Med 2, 1240-3 (1996).
33. Collins, F. S. et al. New goals for the U.S. Human Genome Project: 1998-2003. Science 282, 682-9 (1998).
34. Lander, E. S. The new genomics: global views of biology. Science 274, 536-9 (1996).
35. Falconer, D. S. The inheritance of liability to diseases with variable age of onset, with particular reference to diabetes mellitus. Ann Hum Genet. 31, 1-20 (1967).
36. Waterston, R. H. et al. Initial sequencing and comparative analysis of the mouse genome. Nature 420, 520-62 (2002).
37. Cann, H. M. et al. A human genome diversity cell line panel. Science 296, 261-2 (2002).
38. Stephens, M., Smith, N. J. & Donnelly, P. A new statistical method for haplotype reconstruction from population data. Am J Hum Genet 68, 978-89 (2001).
39. Cutler, D. J. et al. High-throughput variation detection and genotyping using microarrays. Genome Res 11, 1913-25 (2001).
40. Thomas, J. W. et al. Parallel construction of orthologous sequence-ready clone contig maps in multiple species. Genome Res 12, 1277-85 (2002).
41. Thomas, J. W. et al. Comparative analyses of multi-species sequences from targeted genomic regions. Nature 424, 788-93 (2003).
42. Dubchak, I. et al. Active conservation of noncoding sequences revealed by three-way species comparisons. Genome Res 10, 1304-6 (2000).
43. Schwartz, S. et al. PipMaker—a web server for aligning two genomic DNA sequences. Genome Res 10, 577-86 (2000).
43. Thomas, J. W. et al. Parallel construction of orthologous sequence-ready clone contig maps in multiple species. Genome Res. 12, 1277-1285 (2002).
44. Dubchak, I et al. Active conservation of noncoding sequences revealed by three-way species comparisons. Genome Res. 10, 1304-1306 (2000).
45. Schwartz, S. et al. PipMaker—a web server for aligning two genomic DNA sequences. Genome Res. 10, 577-586 (2000).

1. A method of identifying a mutation in DNA, comprising:
predicting a genetic interval for a disease;
comparing orthologous sequences to refine a putative functional interval; and
sequencing the putative functional interval subjects to identify mutations.

2. The method of claim 1, further comprising classifying the refined interval into one or more of coding, non-coding, functional and non-functional sequences.

3. The method of claim 2, wherein the further comparing is after comparing orthologous sequences.

4. The method of claim 1, wherein the predicting comprises one or more of transmission disequilibrium tests (TDT), linkage, or association studies.

5. The method of claim 1, wherein the subjects comprise individuals from affected families.

6. The method of claim 1, wherein the subjects comprise affected and unaffected individuals.

7. The method of claim 6, wherein mutations are over-represented in affected subjects as compared to normal subjects.

8. The method of claim 1, wherein the mutation is associated with a multigenic disease.

9. The method of claim 8, wherein the multigenic disease comprise one or more of mental illness, cancer, cardiovascular disease, congenital anomalies, metabolic disorder inc but not limited to diabetes, susceptibility to infection, drug response, or drug tolerance.

10. The method of claim 1, wherein the mutation comprises a variant of RET.

11. The method of claim 10, wherein the RET variant comprises RET+3:T.

12. The method of claim 1, wherein the mutations are one or more of associated with a disease susceptibility, are causative of disease, are contributory to disease,

13. The method of claim 1, wherein the mutation comprises a single nucleotide polymorphism, a multi-nucleotide polymorphism, an insertion, a deletion, a repeat expansion, genomic rearrangements, or segmental amplification.

14-19. (canceled)

20. A method of identifying a diagnostic marker for a disease, comprising:
predicting a genetic interval for a disease;
comparing orthologous sequences to refine the interval; and
sequencing the refined interval in affected and unaffected subjects to thereby identify a diagnostic marker associated with disease susceptibility, wherein the marker is over represented in affected subjects compared to unaffected subjects.

21. The method of claim 20, further comprising classifying the refined interval into one or more of coding, non-coding, functional and non-functional sequences.

22-38. (canceled)

39. A method of identifying a subject having Hirschsprung disease risk comprising detecting in the subject a mutation in the receptor tyrosine kinase RET, wherein a RET+3:T allele is associated with disease risk.

40-41. (canceled)

42. A kit for detecting the presence of HSCR comprising: primers amplifying the mutation and instructions for use.
